(19) United States
(12) Patent Application Publication
(10) Pub. No.: US 2019/0263894 A1
(43) Pub. Date: Aug. 29, 2019

(54) MONOCLONAL ANTIBODY COCKTAILS FOR TREATMENT OF EBOLA INFECTIONS

(71) Applicant: MAPP BIOPHARMACEUTICAL, INC., SAN DIEGO, CA (US)

(72) Inventors: Andrew Hiatt, Hampton, VA (US); Larry Zeitlin, San Diego, CA (US); Kevin Whaley, Del Mar, CA (US); Michael Pauly, Del Mar, CA (US)

(21) Appl. No.: 16/389,646

(22) Filed: Apr. 19, 2019

Related U.S. Application Data
(63) Continuation of application No. 15/146,990, filed on May 5, 2016.
Application No. 15/146,990 is a continuation-in-part of application No. 14/706,910, filed on May 7, 2015, now abandoned.

(51) Int. Cl. C07K 16/10

(52) U.S. Cl. CPC C07K 16/10; C12N 15/8258

(57) ABSTRACT

Antibody variants originating from the monoclonal antibody 13C6, and wherein the N-glycosylation site within the constant region of the heavy chain contains a glycan that is either wild-type or largely devoid of fucose residues, will bind Ebola virus glycoprotein and provide surprising efficacy in treating animals or humans infected with Ebola virus when used in combination with one or more additional anti-Ebola mAbs. Such antibody cocktails are vastly superior to other known monoclonal antibodies or monoclonal antibody combinations in treating animals and humans infected with the Ebola virus.

### CROSS-REFERENCE TO RELATED APPLICATIONS

This application is a continuing application of U.S. patent application Ser. No. 15/146,990, filed May 5, 2016, which claims priority to U.S. patent application Ser. No. 14/706,910, filed May 7, 2015, which are incorporated herein by reference.

### BACKGROUND OF THE INVENTION

Ebola viruses are highly pathogenic and virulent viruses causing rapidly fatal hemorrhagic fever in humans. Cocktails of antibodies comprising two or more mAbs have been found to be more effective in treating infections with the Ebola virus than any individual mAb used alone (1-4). Antibody sequences that enable and optimize the mAb cocktails for treatment of Ebola are disclosed.

A number of conditions and diseases appear to be associated with Ebola and Marburg infections. Ebola virus disease (EVD) and Marburg virus disease (MARVD) displays high viral loads that cause immune and vascular dysregulation. Major symptoms include fever, severe headache, muscle pain, weakness, fatigue, diarrhea, vomiting, abdominal pain and unexplained hemorrhaging.

EVD and MARVD are usually considered severe and deadly illnesses when humans are concerned. EVD and MARVD outbreaks have shown to have a very high fatality rate ranging from 50-90% with a reported occurrence primarily seen near the tropical rainforests of remote villages in Central and West Africa. These viruses are transmitted to people from wild animals and within the human community through human-to-human contact. Natural host for Ebola virus and Marburg virus are not yet conclusively identified but the most probable host appears to be the fruit bats of the Pteropodidae family. Five subspecies of Ebola virus are recognized to date, with Zaire Ebola virus being the most aggressive of all varieties and recording up to 90% mortality. Two subspecies of Marburg have been identified (MARV and RAVV) both having high mortality in humans. All Ebola and Marburg forms are highly contagious and hence have been classed as Category A Priority Pathogens by WHO. Severely ill patients warrant intensive support therapy. Medical workers in affected areas need to undertake extensive measures to prevent contracting the disease. To date, no particular anti-viral therapy has demonstrated effectiveness in Ebola or Marburg virus infection. Also, no vaccine for use in humans is yet approved by the regulatory bodies. If Ebola or Marburg was actually misused as a biological weapon, it could be a serious threat.

### SUMMARY OF THE INVENTION

We have surprisingly found that murine or humanized antibodies, wherein the CDRs originate from mouse monoclonal antibody 13C6 and the framework and other portions of the antibodies are of murine origin or originate from human germ line, and wherein an N-glycosylation site within the constant region of the heavy chain contains a glycan that is either wild-type or largely devoid of fucose residues, will bind Ebola virus glycoprotein and provide surprisingly excellent efficacy in treating animals or humans infected with Ebola virus when used in combination with one or more additional anti-Ebola mAb. Thus, we have a reasonable basis for believing that antibodies of this specificity offer the opportunity to treat, both prophylactically and therapeutically, conditions in humans that are associated with Ebola virus infection including haemorrhage, multi-organ failure and a shock-like syndrome.

Surprisingly, we have discovered that combinations of monoclonal antibodies comprising such a monoclonal antibody 13C6 as well as additional monoclonal antibodies specific to the Ebola glycoprotein are vastly superior to other known monoclonal antibodies or monoclonal antibody combinations in treating animals and humans infected with the Ebola virus.

According to a first aspect of the invention, there is provided a monoclonal antibody variable region comprising an amino acid sequence deduced from the heavy chain amino acid sequence of the 13C6 monoclonal antibody SEQ ID NO: 1 and the light chain variable region amino acid sequence SEQ ID NO: 2 as well as variants of these sequence that improve the effectiveness, stability, and solubility of the 13C6 antibody.

According to a second aspect of the invention, there is provided a method of preparing a chimeric antibody comprising: providing an expression vector comprising a nucleic acid molecule encoding a constant region domain of a human light chain or heavy chain genetically linked to a nucleic acid encoding a light chain variable region selected from the group consisting of the 13C6 heavy and light chains and variants of those sequences; expressing the expression vector in a suitable host; and recovering the chimeric antibody from said host.

According to a third aspect of the invention, there is provided a method of preparing recombinant antibodies comprising:

providing a nucleotide sequence selected from the group consisting of the 13C6 heavy chain nucleotide sequence SEQ ID NO: 3 and the light chain nucleotide sequence SEQ ID NO: 4 as well as variants of these sequence that improve the effectiveness, stability, and solubility of the 13C6 antibody, and modifying said nucleic acid sequence such that at least one but fewer than about 30 of the amino acid residues encoded by said nucleic acid sequence has been changed or deleted without disrupting antigen binding of said peptide; and expressing and recovering said modified nucleotide sequence;

providing a nucleotide sequence selected from the group consisting of the 2G4 heavy chain nucleotide sequence SEQ ID NO: 5 and the light chain sequence SEQ ID NO: 6 as well as variants of these sequence that improve the effectiveness, stability, and solubility of the 2G4 antibody, and modifying said nucleic acid sequence such that at least one but fewer than about 30 of the amino acid residues encoded by said nucleic acid sequence has been changed or deleted without disrupting antigen binding of said peptide; and expressing and recovering said modified nucleotide sequence; and

providing a nucleotide sequence selected from the group consisting of the 4G7 heavy chain nucleotide sequence SEQ ID NO: 7 and the light chain sequence SEQ ID NO: 8 as well as variants of these sequence that improve the effectiveness, stability, and solubility of the 4G7 antibody, and modifying said nucleic acid sequence such that at least one but fewer than about 30 of the amino acid residues encoded by said nucleic acid sequence has been changed or deleted without disrupting antigen binding of said peptide; and expressing and recovering said modified nucleotide sequence. Also part of the invention are polynucleotide sequences that encode the murine, variant, and humanized antibodies or fragments thereof disclosed above, vectors comprising the polynucleotide sequences encoding the humanized antibodies or fragments thereof, host cells transformed with the vectors or incorporating the polynucleotides that express the humanized antibodies or fragments thereof, pharmaceutical formulations of the humanized antibodies and fragments thereof disclosed herein, and methods of making and using the same.

The advantages of the present variant and humanized antibodies over the original murine mAb include more reliable manufacturability, less batch-to-batch variability in glycosylation, greater stability, less aggregation and comparable or higher potency than the original mAb. This will permit lower doses to give equivalent results. Administration of an antibody of this invention in vivo is capable of neutralizing Ebola viruses and providing reduction in the Ebola infectivity such that the infected immune system is potentially capable of recovering from EVD.

The invention also includes methods of using the 13C6 mAb as well as humanized and other variants to treat and to prevent conditions characterized by EVD, which method comprises administering, preferably systemically, to a human in need of such treatment a therapeutically or prophylactically effective amount of the 13C6 antibodies, or immunologically reactive fragments thereof, either alone or in combination with other anti-Ebola mAbs. The invention also includes methods of using the MR191 mAb as well as variants of MR191 to treat and to prevent conditions characterized by MARVD, which method comprises administering, preferably systemically, to a human in need of such treatment a therapeutically or prophylactically effective amount of the MR191 antibody, or immunologically reactive fragments thereof, either alone or in combination with other anti-MARV mAbs.

Thus, it is one embodiment of the present invention to provide a composition for the treatment of Ebola, the composition comprising: a therapeutically effective combination of i.) a first monoclonal antibody comprising a light chain variable region comprising an amino acid sequence deduced from the nucleic acid molecule as set forth in SEQ ID NO: 4, therapeutically effective mutations, and humanized variants thereof, and a heavy chain variable region comprising an amino acid sequence deduced from the nucleic acid molecule as set forth in SEQ. ID NO: 3, therapeutically effective mutations, and humanized variants thereof, ii.) a second monoclonal antibody comprising a light chain variable region comprising an amino acid sequence deduced from the nucleic acid molecule as set forth in SEQ ID NO: 6, therapeutically effective mutations, and humanized variants thereof, and a heavy chain variable region comprising an amino acid sequence deduced from the nucleic acid molecule as set forth in SEQ. ID NO: 5, therapeutically effective mutations, and humanized variants thereof, and iii.) a third monoclonal antibody comprising a light chain variable region comprising an amino acid sequence deduced from the nucleic acid molecule as set forth in SEQ ID NO: 8, therapeutically effective mutations, and humanized variants thereof, and a heavy chain variable region comprising an amino acid sequence deduced from the nucleic acid molecule as set forth in SEQ. ID NO: 7, therapeutically effective mutations, and humanized variants thereof.

Such an embodiment may further comprise a pharmaceutically acceptable excipient or carrier.

Alternately, such an embodiment may be a composition wherein at least one of the first, second, and third monoclonal antibodies comprise a predominantly single glycoform.

It is yet another embodiment of the present invention to provide such a composition wherein the predominantly single glycoform comprises the GnGn glycan, galactosylated glycans, or sialylated glycans.

It is still another embodiment of the present invention to provide such a composition wherein the predominantly single glycoform comprises less than 5% fucose or xylose.

It is a second embodiment of the present invention to provide a composition for the treatment of Ebola, the composition comprising: a therapeutically effective combination of i.) a first monoclonal antibody comprising a light chain variable region comprising an amino acid sequence deduced from the nucleic acid molecule as set forth in SEQ ID NO: 4, therapeutically effective mutations, and humanized variants thereof, and a heavy chain variable region comprising an amino acid sequence deduced from the nucleic acid molecule as set forth in SEQ. ID NO: 3, therapeutically effective mutations, and humanized variants thereof, and ii.) a second monoclonal antibody that binds the Ebola glycoprotein; iii.) wherein administration of the composition to patients five days following infection with the Ebola virus results in at least a 70% survival rate.

It is another embodiment of the present invention to provide such a composition, wherein the second monoclonal antibody comprises a light chain variable region comprising an amino acid sequence deduced from the nucleic acid molecule as set forth in SEQ ID NO: 6, therapeutically effective mutations, and humanized variants thereof, and a heavy chain variable region comprising an amino acid sequence deduced from the nucleic acid molecule as set forth in SEQ. ID NO: 5, therapeutically effective mutations, and humanized variants thereof.

It is still another embodiment of the present invention to provide such a composition, wherein the second monoclonal antibody comprises a light chain variable region comprising an amino acid sequence deduced from the nucleic acid molecule as set forth in SEQ ID NO: 8, therapeutically effective mutations, and humanized variants thereof, and a heavy chain variable region comprising an amino acid sequence deduced from the nucleic acid molecule as set forth in SEQ. ID NO: 7, therapeutically effective mutations, and humanized variants thereof.

It is yet another embodiment of the present invention to provide such a composition, wherein the patient is a human.

It is still another embodiment of the present invention to provide such a composition and further comprising: a pharmaceutically acceptable excipient or carrier.

It is yet another embodiment of the present invention to provide such a composition, wherein at least one of the first and second monoclonal antibodies comprise a predominantly single glycoform.

It is still another embodiment of the present invention to provide such a composition wherein the predominantly single glycoform comprises the GnGn glycan, galactosylated glycans, or sialylated glycans.

It is yet another embodiment of the present invention to provide such a composition wherein the predominantly single glycoform comprises less than 5% fucose or xylose.

It is a third embodiment of the present invention to provide a method for the treatment of Ebola infection in a patient, the method comprising: i.) identifying a patient in need of Ebola treatment; and ii.) administering to the patient a therapeutically effective amount of a composition comprising a combination of: a) a first monoclonal antibody comprising a light chain variable region comprising an amino acid sequence deduced from the nucleic acid molecule as set forth in SEQ ID NO: 4, therapeutically effective mutations, and humanized variants thereof, and a heavy chain variable region comprising an amino acid sequence deduced from the nucleic acid molecule as set forth in SEQ. ID NO: 3, therapeutically effective mutations, and humanized variants thereof, b) a second monoclonal antibody comprising a light chain variable region comprising an amino acid sequence deduced from the nucleic acid molecule as set forth in SEQ ID NO: 6, therapeutically effective mutations, and humanized variants thereof, and a heavy chain variable region comprising an amino acid sequence deduced from the nucleic acid molecule as set forth in SEQ. ID NO: 5, therapeutically effective mutations, and humanized variants thereof, and c) a third monoclonal antibody comprising a light chain variable region comprising an amino acid sequence deduced from the nucleic acid molecule as set forth in SEQ ID NO: 8, therapeutically effective mutations, and humanized variants thereof, and a heavy chain variable region comprising an amino acid sequence deduced from the nucleic acid molecule as set forth in SEQ. ID NO: 7, therapeutically effective mutations, and humanized variants thereof.

It is another embodiment of the present invention to provide such a method, wherein the patient is a human.

It is yet another embodiment of the present invention to provide such a method, wherein the therapeutically effective composition further comprises a pharmaceutically acceptable excipient or carrier.

The invention also includes methods of treating EVD or MARVD, comprising administering to the subject an effective amount of the antibodies of the present invention.

The invention also includes use of a fully human or humanized antibody of the present invention for the manufacture of a medicament, including prolonged expression of recombinant sequences of the antibody or antibody fragment in human tissues, for treating, preventing, or reversing EVD or MARVD.

### BRIEF DESCRIPTION OF THE DRAWINGS

FIG. 1: A graph showing post-exposure protection of Ebola Virus infected nonhuman primates with ZMAPP.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned above and hereunder are incorporated herein by reference.

### Definitions

As used herein, “neutralizing antibody” refers to an antibody, for example, a monoclonal antibody (mAb), capable of disrupting a formed viral particle or inhibiting formation of a viral particle or prevention of binding to or infection of mammalian cells by a viral particle.

As used herein, “diagnostic antibody” or “detection antibody” or “detecting antibody” refers to an antibody, for example, a monoclonal antibody, capable of detecting the presence of an antigenic target within a sample. As will be appreciated by one of skill in the art, such diagnostic antibodies preferably have high specificity for their antigenic target.

As used herein, “humanized antibodies” refer to antibodies with reduced immunogenicity in humans.

As used herein, “chimeric antibodies” refer to antibodies with reduced immunogenicity in humans built by genetically linking a non-human variable region to human constant domains.

As used herein, the word “treat” includes therapeutic treatment, where a condition to be treated is already known to be present and prophylaxis—i.e., prevention of, or amelioration of, the possible future onset of a condition.

As used herein, a “therapeutically effective” treatment refers a treatment that is capable of producing a desired effect. Such effects include, but are not limited to, enhanced survival, reduction in presence or severity of symptoms, reduced time to recovery, and prevention of initial infection.

By “antibody” is meant a monoclonal antibody (mAb) per se, or an immunologically effective fragment thereof, such as an Fab, Fab′, or F(ab′)2 fragment thereof. In some contexts, herein, fragments will be mentioned specifically for emphasis; nevertheless, it will be understood that regardless of whether fragments are specified, the term “antibody” includes such fragments as well as single-chain forms. As long as the protein retains the ability specifically to bind its intended target, it is included within the term “antibody.” Also included within the definition “antibody” are single chain forms. Preferably, but not necessarily, the antibodies useful in the invention are produced recombinantly. Antibodies may or may not be glycosylated, though glycosylated. Antibodies are preferred. In a further preferred embodiment, the glycosylated antibodies contain glycans that are largely devoid of fucose. In another preferred embodiment, the glycosylated antibodies contain glycans that are galactosylated. In yet another preferred embodiment, the galactosylated antibodies contain glycans that are sialylated. Antibodies are properly cross-linked via disulfide bonds, as is well known.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Light chains are classified as kappa and lambda. Heavy chains are classified as gamma, mu, alpha, delta, or epsilon, and define the antibody's isotype as IgG, IgM, IgA, IgD and IgE, respectively. Within each isotype, there may be subtypes, such as IgG₁, IgG₂, IgG₃, IgG₄, etc. Within light and heavy chains, the variable and constant regions are joined by a “J” region of about 12 or more amino acids, with the heavy chain also including a “D” region of about 3 or more amino acids. The particular identity of constant region, the isotype, or subtype does not impact the present invention. The variable regions of each light/heavy chain pair form the antibody binding site.

Thus, an intact antibody has two binding sites. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with well known conventions [Kabat “Sequences of Proteins of Immunological Interest” National Institutes of Health, Bethesda, Md., 1987 and 1991; Chothia, et al., J. Mol. Biol. 196:901-917 (1987); Chothia, et al., Nature 342:878-883 (1989)].

By “humanized antibody” is meant an antibody that is composed partially or fully of amino acid sequences derived from a human antibody germline by altering the sequence of an antibody having non-human complementarity determining regions (CDR). A humanized immunoglobulin does not encompass a chimeric antibody, having a mouse variable region and a human constant region. However, the variable region of the antibody and even the CDR are humanized by techniques that are by now well known in the art. The framework regions of the variable regions are substituted by the corresponding human framework regions leaving the non-human CDR substantially intact. As mentioned above, it is sufficient for use in the methods of the invention, to employ an immunologically specific fragment of the antibody, including fragments representing single chain forms. Humanized antibodies have at least three potential advantages over non-human and chimeric antibodies for use in human therapy:

1) Because the effector portion is human, it may interact better with the other parts of the human immune system (e.g., destroy the target cells more efficiently by complement-dependent cytotoxicity (CDC) or antibody-dependent cellular cytotoxicity (ADCC)).

2) The human immune system should not recognize the framework or C region of the humanized antibody as foreign, and therefore the antibody response against such an injected antibody should be less than against a totally foreign non-human antibody or a partially foreign chimeric antibody.

3) Injected non-human antibodies have been reported to have a half-life in the human circulation much shorter than the half-life of human antibodies. Injected humanized antibodies will have a half-life essentially identical to naturally occurring human antibodies, allowing smaller and less frequent doses to be given.

The design of humanized immunoglobulins may be carried out as follows. As to the human framework region, a framework or variable region amino acid sequence of a CDR-providing non-human immunoglobulin is compared with corresponding sequences in a human immunoglobulin variable region sequence collection, and a sequence having a high percentage of identical amino acids is selected. When an amino acid falls under the following category, the framework amino acid of a human immunoglobulin to be used (acceptor immunoglobulin) is replaced by a framework amino acid from a CDR-providing non-human immunoglobulin (donor immunoglobulin):

(a) the amino acid in the human framework region of the acceptor immunoglobulin is unusual for human immunoglobulin at that position, whereas the corresponding amino acid in the donor immunoglobulin is typical for human immunoglobulin at that position; (b) the position of the amino acid is immediately adjacent to one of the CDRs; or (c) any side chain atom of a framework amino acid is within about 5-6 angstroms (center-to-center) of any atom of a CDR amino acid in a three dimensional immunoglobulin model [Queen, et al, Proc. Natl Acad. Sci. USA 86:10029-10033 (1989), and Co, et al., Proc. Natl. Acad. Sci. USA 88, 2869 (1991)]. When each of the amino acid in the human framework region of the acceptor immunoglobulin and a corresponding amino acid in the donor immunoglobulin is unusual for human immunoglobulin at that position, such an amino acid is replaced by an amino acid typical for human immunoglobulin at that position.

### The 13C6 mAb is an Essential Component of Antibody Cocktails for Ebola.

A variety of mAbs are available to create cocktails that are effective in neutralizing the Ebola virus, as has been described (1-4). Complete survival of guinea pigs or non-human primates after Ebola virus infection requires a cocktail of mAbs that includes 13C6 (3).

The CDRs of murine 13C6 have the following amino acid sequences:
light chain CDR1: SEQ ID NO: 9
light chain CDR2: SEQ ID NO: 10
light chain CDR3: SEQ ID NO: 11
heavy chain CDR1: SEQ ID NO: 12
heavy chain CDR2: SEQ ID NO: 13
heavy chain CDR3: SEQ ID NO: 14

Described herein are the 13C6 mAb and a number of variants of the 13C6 mAb that are effective in treating animals and human individuals infected with Ebola virus. Treatment is best accomplished by adding 13C6 to other anti-Ebola mAbs to create a cocktail of two or more mAbs. We have surprisingly found that other anti-Ebola mAbs are not as effective, either alone or in combination, as a cocktail containing 13C6. These cocktails can be tested in non-human primates infected with Ebola virus as described below.

These 13C6 antibodies and variants also appear to have high affinity and avidity to Ebola glycoproteins, which means that they could be used as highly sensitive diagnostic tools.

Humanized variants of 13C6 can include but are not limited to heavy chain FR variants
FR1: SEQ ID NO: 15;
FR2: SEQ ID NO: 16;
FR3: SEQ ID NO: 17;
and light chain FR variants
FR1: SEQ ID NO: 18;
FR2: SEQ ID NO: 19;
FR3: SEQ ID NO: 20;
or any other variant that minimizes the immunogenicity of the antibody in humans and retains antigen binding.

One or more of the sequences described herein comprising or encoding the 13C6 antibody can be subjected to humanization techniques or converted into chimeric human molecules for generating a variant antibody which has reduced immunogenicity in humans. Humanization techniques are well known in the art—see for example U.S. Pat. Nos. 6,309,636 and 6,407,213 which are incorporated herein by reference specifically for their disclosure on humanization techniques. Chimerics are also well known, see for example U.S. Pat. Nos. 6,461,824, 6,204,023, 6,020,153 and 6,120,767 which are similarly incorporated herein by reference. Such techniques can also be applied to antibodies other than 13C6, such as those described herein, to achieve predictable results.

In one embodiment of the invention, chimeric antibodies are formed by preparing an expression vector which comprises a nucleic acid encoding a constant region domain of a human light or heavy chain genetically linked to a nucleic acid encoding a light chain variable region selected from the group consisting of 13C6 and its variants disclosed herein.

Additional variants of 13C6 include but are not limited to mutations in FRs that improve the stability, solubility, and production. These mutations include but are not limited to the heavy chain sequences of SEQ ID NOs: 21-23.

Additional mutations include but are not limited to the light chain sequences of SEQ ID NOs: 24-25.

A naturally occurring mutation in the light chain FR4 has the surprising result that aggregation to high molecular weight (HMW) structures is significantly augmented. This (light chain FR4.1) has the surprising result that aggregation to high molecular weight (HMW) aggregates is significantly minimized.

Light chain FR4.1:
(SEQ ID NO: 26)
FGAGTKLELKR

The heavy chain mutations can be combined with any of the light chain mutations to achieve the desired effect on expression, stability, or solubility when introduced into a host organism. In a preferred embodiment, the host organism for the production of wild-type and mutated sequences of 13C6 is Nicotiana benthamiana.

In another embodiment of the invention, there are provided recombinant antibodies comprising at least one modified variable region, said region selected from the group consisting of 13C6 and its variants in which at least one but fewer than about 30 of the amino acid residues of said variable region has been changed or deleted without disrupting antigen binding.

### MR191 Anti-Margburg Virus mAB

We have surprisingly found that the fully human MR191 anti-Marburg virus mAb can confer 100% protection and post exposure treatment to non-human primates (NHPs). Thus, we have a reasonable basis for believing that this mAb offers the opportunity to treat, both prophylactically and therapeutically, conditions in humans that are associated with MARVD.

The CDRs of MR191 have the following amino acid sequences:

Light chain CDR1:
(SEQ ID NO: 27)
TGSSSNIGAGFDVH
Light chain CDR2:
(SEQ ID NO: 28)
DNNNRPS
Light chain CDR3:
(SEQ ID NO: 29)
QSYDTSLSGPVV
Heavy chain CDR1:
(SEQ ID NO: 30)
GVSISDNSYYWG
Heavy chain CDR2:
(SEQ ID NO: 31)
TISYSGNTYYNPSL
Heavy chain CDR3:
(SEQ ID NO: 32)
QRIVSGFVEWLSKFDY
The FRs of MR191 have the following
amino acid sequences:
Light chain FR1:
(SEQ ID NO: 33)
QSVLTQPPSVSGAPGQRVTISC
Light chain FR2:
(SEQ ID NO: 34)
WYQQLPGTAPKLLIY
Light chain FR3:
(SEQ ID NO: 35)
GVPDRFSGSKSGTSASLAITGLQAEDEADYYC
Light chain FR4:
(SEQ ID NO: 36)
FGGGTKLTVLQPK
Heavy chain FR1:
(SEQ ID NO: 37)
QLQLQESGPGLVKPSETLSLSCTVS
Heavy chain FR2:
(SEQ ID NO: 38)
WIRQPPGKGLEWIG
Heavy chain FR3:
(SEQ ID NO: 39)
KSRVSISGDTSKHQLSLKVSSVTAADTAVYYCAR
Heavy chain FR4:
(SEQ ID NO: 40)
WGQGTLVTVSS

In yet other embodiments, immunoreactive fragments of any of the above-described monoclonal antibodies, chimeric antibodies or humanized antibodies are prepared using means known in the art, for example, by preparing nested deletions using enzymatic degradation or convenient restriction enzymes.

In another embodiment of the invention, there are provided recombinant antibodies comprising at least one modified variable region, said region selected from the group consisting of MR191 and its variants in which at least one but fewer than about 30 of the amino acid residues of said variable region has been changed or deleted without disrupting antigen binding. Preferably, such variants improve the stability, solubility, or production of the MR191.

It is of note that in all embodiments describing preparation of humanized antibodies, chimeric antibodies or immunoreactive fragments of monoclonal antibodies, these antibodies are screened to ensure that antigen binding has not been disrupted. This may be accomplished by any of a variety of means known in the art, but one convenient method would involve use of a phage display library. As will be appreciated by one of skill in the art, as used herein, ‘immunoreactive fragment’ refers in this context to an antibody fragment reduced in length compared to the wild-type or parent antibody which retains an acceptable degree or percentage of binding activity to the target antigen. As will be appreciated by one of skill in the art, what is an acceptable degree will depend on the intended use.

Other sequences are possible for the light and heavy chains for the human or humanized antibodies of the present invention. The immunoglobulins can have two pairs of light chain/heavy chain complexes, at least one chain comprising one or more mouse complementarity determining regions functionally joined to human framework region segments.

The polynucleotides will typically further include an expression control polynucleotide sequence operably linked to the humanized immunoglobulin coding sequences, including naturally-associated or heterologous promoter regions. Preferably, the expression control sequences will be eukaryotic promoter systems in vectors capable of transforming or transfecting eukaryotic host cells, but control sequences for prokaryotic hosts may also be used. Once the vector has been incorporated into the appropriate host cell line, the host cell is propagated under conditions suitable for expressing the nucleotide sequences, and, as desired, the collection and purification of the light chains, heavy chains, light/heavy chain dimers or intact antibodies, binding fragments or other immunoglobulin forms may follow. The nucleic acid sequences of the present invention capable of ultimately expressing the desired humanized antibodies can be formed from a variety of different polynucleotides (genomic or cDNA, RNA, synthetic oligonucleotides, etc.) and components (e.g., V, J, D, and C regions), using any of a variety of well-known techniques. Joining appropriate genomic and synthetic sequences is a common method of production, but cDNA sequences may also be utilized.

Human constant region DNA sequences can be isolated in accordance with well-known procedures from a variety of human cells, but preferably from immortalized B— cells. Suitable source cells for the polynucleotide sequences and host cells for immunoglobulin expression and secretion can be obtained from a number of sources well known in the art.

In addition to the humanized immunoglobulins specifically described herein, other “substantially homologous” modified immunoglobulins can be readily designed and manufactured utilizing various recombinant DNA techniques well known to those skilled in the art. For example, the framework regions can vary from the native sequences at the primary structure level by several amino acid substitutions, terminal and intermediate additions and deletions, and the like. Moreover, a variety of different human framework regions may be used singly or in combination as a basis for the humanized immunoglobulins of the present invention. In general, modifications of the genes may be readily accomplished by a variety of well-known techniques, such as site-directed mutagenesis.

Alternatively, polypeptide fragments comprising only a portion of the primary antibody structure may be produced, which fragments possess one or more immunoglobulin activities (e.g., complement fixation activity). These polypeptide fragments may be produced by proteolytic cleavage of intact antibodies by methods well known in the art, or by inserting stop codons at the desired locations in vectors using site-directed mutagenesis, such as after CHI to produce Fab fragments or after the hinge region to produce F(ab′)2 fragments. Single chain antibodies may be produced by joining NL and NH with a DNA linker. As stated previously, the polynucleotides will be expressed in hosts after the sequences have been operably linked to (i.e., positioned to ensure the functioning of) an expression control sequence. These expression vectors are typically replicable in the host organisms either as episomes or as an integral part of the host chromosomal DNA. Commonly, expression vectors will contain selection markers, e.g., tetracycline or neomycin, to permit detection of those cells transformed with the desired DNA sequences. Expression vectors for these cells can include expression control sequences, such as an origin of replication, a promoter, an enhancer, and necessary processing information sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, cytomegalovirus and the like. The vectors containing the polynucleotide sequences of interest (e.g., the heavy and light chain encoding sequences and expression control sequences) can be transferred into the host cell by well-known methods, which vary depending on the type of cellular host. A variety of hosts may be employed to express the antibodies of the present invention using techniques well known in the art. Mammalian tissue cell culture is preferred, especially using, for example, CHO, COS, Syrian Hamster Ovary, HeLa, myeloma, transformed B-cells, human embryonic kidney, or hybridoma cell lines.

It is of note that as discussed herein, any of the described antibodies or humanized variants thereof may be formulated into a pharmaceutical treatment for providing passive immunity for individuals suspected of or at risk of developing hemorrhagic fever comprising a therapeutically effective amount of said antibodies. The pharmaceutical preparation may include a suitable excipient or carrier. See, for example, Remington: The Science and Practice of Pharmacy, 1995, Gennaro ed. As will be apparent to one knowledgeable in the art, the total dosage will vary according to the weight, health and circumstances of the individual as well as the efficacy of the antibodies.

In a preferred embodiment, the antibodies of the present invention are produced in plants. Consistent manufacturing and quality control of an antibody drug substance with pre-defined specifications is a key feature for the successful development of antibody mixtures for human therapeutic use [Zwick, M. B., et al. (2001). “Neutralization synergy of human immunodeficiency virus type 1 primary isolates by cocktails of broadly neutralizing antibodies.” J Virol 75(24): 12198-12208; Pedersen, M. W., et al. (2010). “Sym004: a novel synergistic anti-epidermal growth factor receptor antibody mixture with superior anticancer efficacy.” Cancer Res 70(2): 588-597; Doria-Rose, N. A., et al. (2012). “HIV-1 neutralization coverage is improved by combining monoclonal antibodies that target independent epitopes.” J Virol 86(6): 3393-3397; Klein, F., et al. (2012). “HIV therapy by a combination of broadly neutralizing antibodies in humanized mice.” Nature 492(7427): 118-122; Davies, N. L., et al. (2013). “A mixture of functionally oligoclonal humanized monoclonal antibodies that neutralize Clostridium difficile TcdA and TcdB with high levels of in vitro potency shows in vivo protection in a hamster infection model.” Clin Vaccine Immunol 20(3): 377-390; (5)]. Multi-Ab cGMP processes have been developed where numerous mAbs can be incorporated into a single drug substance (Frandsen, T. P., et al. (2011). “Consistent manufacturing and quality control of a highly complex recombinant polyclonal antibody product for human therapeutic use.” Biotechnol Bioeng 108(9): 2171-2181). These cGMP processes were developed for CHO cell manufacturing and consisted of a defined number of mAbs manufactured from a polyclonal cell bank [Bregenholt, S. and J. Haurum (2004). “Pathogen-specific recombinant human polyclonal antibodies: biodefence applications.” Expert Opin Biol Ther 4(3): 387-396; Bregenholt, S., et al. (2006). “Recombinant human polyclonal antibodies: A new class of therapeutic antibodies against viral infections.” Curr Pharm Des 12(16): 2007-2015; Meijer, P. J., et al. (2006). “Isolation of human antibody repertoires with preservation of the natural heavy and light chain pairing.” J Mol Biol 358(3): 764-772; Tolstrup, A. B., et al. (2006). “Development of recombinant human polyclonal antibodies for the treatment of complex human diseases.” Expert Opin Biol Ther 6(9): 905-912; Wiberg, F. C., et al. (2006). “Production of target-specific recombinant human polyclonal antibodies in mammalian cells.” Biotechnol Bioeng 94(2): 396-405; Nielsen, L. S., et al. (2010). “Single-batch production of recombinant human polyclonal antibodies.” Mol Biotechnol 45(3): 257-266]. The expression platform for CHO utilized a site-specific integration technology necessary for stable individual CHO cell lines producing each of the antibodies. These were expanded under GMP conditions and subsequently mixed in equal numbers to generate a polyclonal Master Cell Bank (pMCB). One problem with the CHO system for production of multi-mAb drug substance is the time it takes to generate individual cell lines. This is an inherent problem of animal cell production since numerous variables require optimization [Li, F., et al. (2010). “Cell culture processes for monoclonal antibody production.” MAbs 2(5): 466-479]. These include (a) establishing cell lines capable of producing mAbs at levels that ensure low operating cost; (b) establishing culture media and bioreactor culture conditions for productivity and quality specifications; (c) employing appropriate on-line and off-line sensors for information that enhances process control; and (d) a good understanding of culture performance at different scales to ensure smooth scale-up. Additionally, for a multi-mAb product, engineering cell lines with defined sites of genomic integration of mAb vectors is required in order to ensure consistent production of individual mAbs in the pMCB. This process of selection for stability and high level production can take many months [Li, F., et al. (2010)].

The plant-based Rapid Antibody Manufacturing Platform (RAMP) system does not require any of the optimization steps that are needed for CHO-based mAb production (13). This is largely because genomic integration of mAb genes does not occur in RAMP. Instead, Agrobacterium delivery of viral pro-vectors introduces mAb genes that remain extra-nuclear, allowing for robust production from virus-derived DNA sequences. In addition, the ability to scale up production is far more predictable than CHO because the growth conditions are invariant, relying only on constant temperature, light, water, and simple nutrients. Moreover, the ability to alter and test mAb sequences prior to production runs occurs at a much faster pace since mAb expression takes only two weeks from mAb-encoding DNA to raw material containing functional antibody (13,32). For production of a multi-mAb drug substance, each mAb would be separately infected into batches of Nicotiana plants that would then be mixed to form the equivalent of a polyclonal plant bank. However, none of the plant cells in the individual or the combined plant populations contain viable, propagating cells. The entire process depends entirely on a single Agrobacterium per mAb that is used for infection and not genome integration. Agrobacteria are grown overnight and used for only a five minute plant inoculation. The consistency of the infection process has been well established (Werner, S., et al. (2011). “High-level recombinant protein expression in transgenic plants by using a double-inducible viral vector.” Proc Natl Acad Sci USA 108(34): 14061-14066; Gleba, Y. Y., et al. (2013). “Plant Viral Vectors for Delivery by Agrobacterium.” Curr Top Microbiol Immunol). The Nicotiana plants are in effect the medium for expressing the antibody-encoding pro-vectors transferred by Agrobacterium.

Another important aspect of the adaptable RAMP system is its ability to be easily sized to an appropriate GMP production scale. RAMP has been shown to be a linearly scalable system; the facilities at KBP have demonstrated the capability of GMP production from small batch production up to 3000 kg/hr of biomass, which can then processed in a relatively small GMP compliant clean room facility. Thus, the platform can produce a wide variety of proteins suitable for human therapeutics in high yield and in a very short period of time—plus, the process is both easily scaled and operable to GMP. All these benefits make RAMP an extremely adaptable platform technology. Thus, the platform can produce mAbs suitable for human therapeutics in high yield and in a short period of time. Further, the FDA is gaining significant experience with plant-derived biologics (Table 1) such as those being developed here.

TABLE 1
Plant-derived biologics are gaining regulatory acceptance

| Company | Plant system | Product | Clinical stage |
|---|---|---|---|
| Protalix/Pfizer | Carrot | Enzyme replacement | Licensed |
| ASTI | Strawberry | Canine interferon | Licensed |
| | | alpha | |
| Medicago | Nicotiana | Influenza vaccine | Phase 2 |
| Meristem | Corn | Enzyme | Phase 2 |
| Planet | Nicotiana | Anti-caries mAb | Phase 2 |
| Icon/Bayer | Nicotiana | mAb cancer vaccine | Phase 1 |
| Large Scale | Nicotiana | scFv cancer vaccine | Phase 1 |
| Biology | | | |
| iBio | Nicotiana | Influenza vaccine | Phase 1 |
| Monsanto | Corn | Cancer therapy | Phase 1 |

Finally, it is also anticipated that significant cost-savings in the final commercial product can be realized. KBP estimates that the breast cancer drug product at commercial scale will cost less than $50/g—manufacturing costs for mAbs produced in CHO or NSO are typically described as ranging from $200-4000/g (Farid, S. S. (2007). “Process economics of industrial monoclonal antibody manufacture.” J Chromatogr B Analyt Technol Biomed Life Sci 848(1): 8-18). In addition, the production facility cost for RAMP is far less than for any animal cell-based facility (49).

The enhancement of immune effector functions has been proposed as a potential strategy for increasing the efficacy of therapeutic antibodies (Listinsky, J. J., et al. (2011). “The emerging importance of alpha-L-fucose in human breast cancer: a review.” Am J Transl Res 3(4): 292-322). In particular, removal of fucose from mAb glycans has been shown to enhance the efficacy of numerous mAbs (Clynes, R. A., et al. (2000). “Inhibitory Fc receptors modulate in vivo cytotoxicity against tumor targets.” Nat Med 6(4): 443-446; Shields, R. L., et al. (2002). “Lack of fucose on human IgG1 N-linked oligosaccharide improves binding to human Fcgamma RIII and antibody-dependent cellular toxicity.” J Biol Chem 277(30): 26733-26740; Shinkawa, T., et al. (2003). “The absence of fucose but not the presence of galactose or bisecting N-acetylglucosamine of human IgG1 complex-type oligosaccharides shows the critical role of enhancing antibody-dependent cellular cytotoxicity.” J Biol Chem 278(5): 3466-3473; Okazaki, A., et al. (2004). “Fucose depletion from human IgG1 oligosaccharide enhances binding enthalpy and association rate between IgG1 and FcgammaRIIIa.” J Mol Biol 336(5): 1239-1249; (33)).

Mapp has expressed over 50 different mAbs using the RAMP platform, the majority of which are against infectious disease antigens, and to date, all have been identical to those produced in mammalian cell culture when analyzed by a variety of in vitro and in vivo assays. In fact, glycosylation has historically been the only practical difference between mAbs produced in mammalian cell culture and in plant tissue. Wild-type N. benthamiana glycosylates proteins differently than mammalian expression systems. N. benthamiana, like other plants, produces the same core glycan as found in mammals, but uses xylose (which generally is not found in mammals) and fucose in a non-mammalian linkage (alpha 1,3). Because of the potential for the novel plant glycans to affect pharmacokinetics as well as immunogenicity in humans, it is highly desirable to produce mAbs in plants that have been modified to generate more mammalian-like glycans. The resulting glycans are more homogeneous than FDA-approved mAbs produced in mammalian cell culture (top three rows). This knockout line will be used to manufacture the breast cancer Abs.

Enhanced ADCC Activity.

We have previously demonstrated that Nicotiana benthamiana plants that have been engineered to have greatly reduced xylosyl- and fucosyltransferase activity (XF) result in antibody glycans that are homogeneous compared to CHO produced mAbs, are capable of being further modified by sialylation to be human-like in structure, and demonstrate enhanced in vivo efficacy in different models of viral infection ((33, 50); Zeitlin, L., et al. (2013). “Prophylactic and therapeutic testing of Nicotiana-derived RSV-neutralizing human monoclonal antibodies in the cotton rat model.” MAbs 5(2): 263-269). For these reasons, all of the mAbs in the current proposal will be produced in the XF— plant line to produce the predominantly GnGn glycan structure. It has been well established that trastuzumab engineered to have glycans that are devoid of fucose has significantly improved progression-free survival when compared with conventional trastuzumab in treating preclinical models of HER2-amplified breast cancer (Junttila, T. T., et al. (2010). “Superior in vivo efficacy of afucosylated trastuzumab in the treatment of HER2-amplified breast cancer.” Cancer Res 70(11): 4481-4489).

Once expressed, the antibodies can be purified according to standard procedures. Substantially pure immunoglobulins of at least about 90 to 95% homogeneity are preferred, and 98 to 99% or more homogeneity most preferred, for pharmaceutical uses. Once purified, partially or to homogeneity as desired, the polypeptides may then be used therapeutically or prophylactically, as directed herein.

In another embodiment of the invention, there are provided glycoengineered variants of 13C6 and other monoclonal antibodies that contain predominantly a single glycoform. These glycans can be GnGn (GlcNAc₂-Man₃-GlcNAc₂), mono- or di-galactosylated (Gal_{(1/2)}-GlcNAc₂-Man₃-GlcNAc₂), mono- or di-sialylated (NaNa_{(1,2)}-Gal_{(1/2)}-GlcNAc₂-Man₃-GlcNAc₂) containing little or no fucose or xylose. A predominantly single glycoform is any glycoform that represents more than half (e.g. greater than 50%, 60%, 70%, 80%, 90%, 95%, 96%, 97%, 98%, 99%) of all glycoforms present in the antibody solution.

The RAMP system has been used for glycoengineering of antibodies, antibody fragments, idiotype vaccines, enzymes, and cytokines. Dozens of antibodies have been produced in the RAMP system by Mapp (5, 6) and others (7, 8). These have predominantly been IgGs but other isotypes, including IgM (9, 10), have been glycoengineered. Glycoengineering has also been extended to human enzymes in the RAMP system (11, 12). Since the RAMP system has a rapid turn-around time from Agrobacterium infection to harvest and purification (13) patient specific idiotype vaccines have been used in clinical trials for non-Hodgkins lymphoma (7).

For glycoengineering, recombinant Agrobacterium containing a 13C6 mAb cDNA, or other mAb cDNA, is used for infection of N. benthamiana in combination with the appropriate glycosylation Agrobacteria to produce the desired glycan profile. For wild-type glycans (i.e. native, plant-produced glycosylation) wild-type N. benthamiana is inoculated with only the Agrobacterium containing the anti-M2e cDNA. For the GnGn glycan, the same Agrobacterium is used to inoculate plants that contain little or no fucosyl or xylosyl transfrases (XF plants). For galactosylated glycans, XF plants are inoculated with the Agrobacterium containing the 13C6 cDNA as well as an Agrobacterium containing the cDNA for β-1,4-galactosyltransferase expression contained on a binary Agrobacterium vector to avoid recombination with the TMV and PVX vectors (14). For sialylated glycans, six additional genes are introduced in binary vectors to reconstitute the mammalian sialic acid biosynthetic pathway. The genes are UDP-N-acetylglucosamine 2-epimerase/N-acetylmannosamine kinase, N-acetylneuraminic acid phosphate synthase, CMP-N-acetylneuraminic acid synthetase, CMP-NeuAc transporter, β-1,4-galactosylatransferase, and α2,6-sialyltransferase (14).

Glycanalysis of glycoengineered mAbs involved release of N-linked glycans by digestion with N-glycosidase F (PNGase F), and subsequent derivatization of the free glycan is achieved with anthranilic acid (2-AA). The 2-AA-derivatized oligosaccharide is separated from any excess reagent via normal-phase HPLC. The column is calibrated with 2-AA-labeled glucose homopolymers and glycan standards. The test samples and 2-AA-labeled glycan standards are detected fluorometrically. Glycoforms are assigned either by comparing their glucose unit (GU) values with those of the 2-AA-labeled glycan standards or by comparing with the theoretical GU values (15). Confirmation of glycan structure was accomplished with LC/MS.

While the RAMP system is an effective method of producing various glycoengineered and wild-type mABs, it will be recognized that other expression systems may be used to accomplish the same result. For example, mammalian cell lines (such as CHO or NSO cells [Davies, J., Jiang, L., Pan, L. Z., LaBarre, M. J., Anderson, D., and Reff, M. 2001. Expression of GnTIII in a recombinant anti-CD20 CHO production cell line: Expression of antibodies with altered glycoforms leads to an increase in ADCC through higher affinity for FCyRIII. Biotechnol Bioeng 74:288-294]), yeast cells (such as Pichia pastoris [Gerngross T. Production of complex human glycoproteins in yeast. Adv Exp Med Biol. 2005; 564]) and bacterial cells (such as E. Co/i) have been used produce such mABs.

TABLE 2
Glycananlysis of 2G4, 13C6FR1, and 4G7 antibodies produced using the RAMP system.

| | FLR | | | | | | |
|---|---|---|---|---|---|---|---|
| | RT | FLR Peak Area | | | % Abundance | | |
| Isoforms^{a} | (min) | C2G4 | C13C6FR1 | C4G7 | C2G4 | C13C6FR1 | C4G7 |
| unknown1 | 14.5 | 18864 | 11275 | 17640 | 0.8 | N/A | 0.6 |
| unknown2 | 15.8 | 20601 | 25759 | 28937 | 0.9 | 0.9 | 1.0 |
| unknown3 | 16.1 | 12637 | 15906 | 17746 | 0.6 | 0.5 | 0.6 |
| G0-GlcNAc | 17.7 | 27255 | 31183 | 20977 | 1.2 | 1.1 | 0.8 |
| G0 | 20.5 | 1988075 | 2358378 | 2584314 | 88.4 | 80.8 | 92.6 |
| G0-GlcNAc + Man | 21.9 | 17977 | 22786 | 10420 | 0.8 | 0.8 | N/A |
| Man5 | 23.4 | 31534 | 44372 | 11974 | 1.4 | 1.5 | N/A |
| G1(a) | 24.0 | 24183 | 11683 | 3936 | 1.1 | 0.4 | 0.1 |
| G1(b) | 24.4 | 25493 | 15866 | 12213 | 1.1 | 0.5 | N/A |
| G0-GlcNAc + 2Man | 25.6 | 21026 | 32958 | 4934 | 0.9 | 1.1 | N/A |
| Man6 | 27.2 | 5941 | 20789 | 1894 | N/A | 0.7 | N/A |
| Man7 | 30.6 | 17435 | 77267 | 13758 | 0.8 | 2.6 | 0.5 |
| Man8 | 33.9 | 22831 | 162345 | 37658 | 1.0 | 5.6 | 1.3 |
| Man9 | 36.3 | 15376 | 88381 | 25573 | 0.7 | 3.0 | 0.9 |

^{a}Only detected glycans with FLR peak area ≥0.5% relative to the most abundant glycan (G0) are reported in this table.

As illustrated in Table 2, the RAMP system is effective for producing monoclonal antibodies that have little or no fucose or xylose (for example less than 5% or less than 1% fucose or xylose). Isoforms containing fucose, xylose, or both could only be represented in the three “unknown” catagories of Table 2.

The 13C6 or MR191 antibodies or variants (including immunologically reactive fragments) are administered to a subject at risk for or exhibiting EVD or MARCD-related symptoms using standard administration techniques, preferably peripherally (i.e. not by administration into the central nervous system) by intravenous, intraperitoneal, subcutaneous, pulmonary, transdermal, intramuscular, intranasal, buccal, sublingual, or suppository administration. Although the antibodies may be administered directly into the ventricular system, spinal fluid, or brain parenchyma, and techniques for addressing these locations are well known in the art, it is not necessary to utilize these more difficult procedures. The antibodies of the invention are effective when administered by the more simple techniques that rely on the peripheral circulation system. The pharmaceutical compositions for administration are designed to be appropriate for the selected mode of administration, and pharmaceutically acceptable excipients such as, buffers, surfactants, preservatives, solubilizing agents, isotonicity agents, stabilizing agents and the like are used as appropriate. Remington's Pharmaceutical Sciences, Mack Publishing Co., Easton Pa., latest edition, incorporated herein by reference, provides a compendium of formulation techniques as are generally known to practitioners.

The concentration of the humanized antibody in formulations from as low as about 0.1% to as much as 15 or 20% by weight and will be selected primarily based on fluid volumes, viscosities, and so forth, in accordance with the particular mode of administration selected. Thus, a pharmaceutical composition for injection could be made up to contain in 1 mL of phosphate buffered saline from 1 to 100 mg of the humanized antibody of the present invention. The formulation could be sterile filtered after making the formulation, or otherwise made microbiologically acceptable. A typical composition for intravenous infusion could have a volume as much as 250 mL of fluid, such as sterile Ringer's solution, and 1-100 mg per mL, or more in antibody concentration. Therapeutic agents of the invention can be frozen or lyophilized for storage and reconstituted in a suitable sterile carrier prior to use. Lyophilization and reconstitution can lead to varying degrees of antibody activity loss (e.g. with conventional immune globulins, IgM antibodies tend to have greater activity loss than IgG antibodies). Dosages may have to be adjusted to compensate. The pH of the formulation will be selected to balance antibody stability (chemical and physical) and comfort to the patient when administered.

Generally, pH between 4 and 8 is tolerated. Although the foregoing methods appear the most convenient and most appropriate for administration of proteins such as humanized antibodies, by suitable adaptation, other techniques for administration, such as transdermal administration and oral administration may be employed provided proper formulation is designed. In addition, it may be desirable to employ controlled release formulations using biodegradable films and matrices, or osmotic mini-pumps, or delivery systems based on dextran beads, alginate, or collagen. In summary, formulations are available for administering the antibodies of the invention and are well-known in the art and may be chosen from a variety of options. Typical dosage levels can be optimized using standard clinical techniques and will be dependent on the mode of administration and the condition of the patient.

While the preferred embodiments of the invention are described herein, it will be recognized and understood that various modifications may be made therein, and the appended claims are intended to cover all such modifications which may fall within the spirit and scope of the invention. The following examples are intended to illustrate, but not limit, the invention.

### 13C6 for Ebola Treatment

Without an approved vaccine or treatment, Ebola outbreak management has been limited to palliative care and barrier methods to prevent transmission. These approaches, however, have yet to end the 2014 outbreak of Ebola after its prolonged presence in West Africa. Here we show that a combination of monoclonal antibodies (ZMAPP), optimized from two previous antibody cocktails, is able to rescue 100% of rhesus macaques when treatment is initiated up to 5 days post-challenge. High fever, viremia, and abnormalities in blood count and chemistry were evident in many animals before ZMAPP intervention. Advanced disease, as indicated by elevated liver enzymes, mucosal hemorrhages and generalized petechia could be reversed, leading to full recovery. ELISA and neutralizing antibody assays indicate that ZMAPP is cross-reactive with the Guinean variant of Ebola. ZMAPP currently exceeds all previous descriptions of efficacy with other therapeutics, and results warrant further development of this cocktail for clinical use.

Ebola virus (EBOV) infections cause severe illness in humans, and after an incubation period of 3 to 21 days, patients initially present with general flu-like symptoms before a rapid progression to advanced disease characterized by hemorrhage, multi-organ failure and a shock-like syndrome (16). In the spring of 2014, a new EBOV variant emerged in the West African country of Guinea (17), an area in which EBOV has not been previously reported. Despite a sustained international response from local and international authorities including the Ministry of Health (MOH), World Health Organization (WHO) and Médecins Sans Frontières (MSF) since March 2014, the outbreak has yet to be brought to an end after five months. As of 15 Aug. 2014, there are 2127 total cases and 1145 deaths spanning Guinea, Sierra Leone, Liberia and Nigeria (18). So far, this outbreak has set the record for the largest number of cases and fatalities, in addition to geographical spread (19). Controlling an EBOV outbreak of this magnitude has proven to be a challenge and the outbreak is predicted to last for at least several more months (20). In the absence of licensed vaccines and therapeutics against EBOV, there is little that can be done for infected patients outside of supportive care, which includes fluid replenishment, administration of antivirals, and management of secondary symptoms (21) (22). With overburdened personnel, and strained local and international resources, experimental treatment options cannot be considered for compassionate use in an orderly fashion at the moment. However, moving promising strategies forward through the regulatory process of clinical development has never been more urgent.

Over the past decade, several experimental strategies have shown promise in treating EBOV-challenged nonhuman primates (NHPs) after infection. These include recombinant human activated protein C (rhAPC) (23), recombinant nematode anticoagulant protein c2 (rNAPc2) (24), small interfering RNA (siRNA) (25), positively-charged phosphorodiamidate morpholino oligomers (PMOplus) (26), the vesicular stomatitis virus vaccine (VSVΔG-EBOVGP)(27), as well as the monoclonal antibody (mAb) cocktails MB-003 (consisting of human or human-mouse chimeric mAbs c13C6, h13F6 and c6D8) (28) and ZMAb (consisting of murine mAbs m1H3, m2G4 and m4G7) (29) (U.S. Pat. No. 8,513,391). Of these, only the antibody-based candidates have demonstrated substantial benefits in NHPs when administered greater than 24 hours past EBOV exposure. Follow-up studies have shown that MB-003 is partially efficacious when administered therapeutically after the detection of two disease “triggers” (30), and ZMAb combined with an adenovirus-based adjuvant provides full protection in rhesus macaques when given up to 72 hours after infection (31).

Our objective was to develop a therapeutic superior to both MB-003 and ZMAb, which could be utilized for outbreak patients, primary health-care providers, as well as high-containment laboratory workers in the future. The study aimed to first identify an optimized antibody combination derived from MB-003 and ZMAb components, before determining the therapeutic limit of this mAb cocktail in a subsequent experiment. In order to extend the antibody half-life in humans and to facilitate clinical acceptance, the individual murine antibodies in ZMAb were first chimerized with human constant regions (cZMAb). The cZMAb components were then produced in Nicotiana benthamiana (32), using the large-scale, cGMP-compatible Rapid Antibody Manufacturing Platform (RAMP) and magnICON vectors that currently also manufactures the individual components of cocktail MB-003, before efficacy testing in animals.

### EXAMPLES

### Example 1: 13C6 Bioprocessing

The 2014/2015 Ebola virus disease (EVD) in West Africa was the largest outbreak in history (51). This Ebola virus outbreak appears to have been caused by the Zaire species of the virus, which can have fatality rates up to 90% (24). EVD displays high viral loads that cause immune and vascular dysregulation. Major symptoms include fever, severe headache, muscle pain, weakness, fatigue, diarrhea, vomiting, abdominal pain and unexplained hemorrhaging.

Currently there are no licensed vaccines or medicines for the treatment of EVD. Infected patients are treated with supportive-care rehydration of oral or intravenous fluids while maintaining oxygen and blood pressure levels. Therapeutic strategies targeting EVD include recombinant human activated protein, recombinant nematode anticoagulant protein c2, small interfering RNA, positively-charged phosphorodiamidate morpholino oligomers, the vesicular stomatitis virus vaccine, and monoclonal antibody (mAb) cocktails (3, 23-31). Of these strategies the monoclonal antibody cocktail ZMapp™, in particular, has shown promise in non-human primate studies (26) and was used to treat 7 patients during the 2014 outbreak of EVD. (52). ZMapp™ consists of three antibodies: c13C6FR1, c2G4, and c4G7 which have been expressed in a tobacco system, Nicotiana benthamiana. The c13C6FR1 mAb is comprised of light chain FR1.1 (SEQ ID NO: 22) and heavy chain FR1.3 (SEQ ID NO: 23). In some experiments described below the light chain FR4 contains K at position 148 (c13C6FR1+K) (SEQ ID NO: 26). The constant regions are human (IgG1-Kappa).

By the fall of 2014, it was clear that there was insufficient ZMapp™ stockpiled to treat the number of patients infected with the Ebola virus. In addition, the scale of the tobacco production system could not support the demand of an ongoing epidemic. Rapid scale-up of antibodies in tobacco plants can be challenging based on expression levels and limited sites for production. A high expressing CHO production system, on the other hand, could support rapid generation of large quantities of drug product either by increasing bioreactor scale or with production in multiple facilities. Scalable antibody production in CHO cells has the potential to produce enough material to meet demand of future EVD outbreaks. (53). Efforts are currently underway to produce ZMapp™ anti-Ebola antibody sequences in large scale tobacco and CHO cell production systems.

Antibody constant and variable domain sequences are known to impact binding specificity and biological activities such as pharmacokinetics and effector functions, but these sequences can also significantly impact antibody manufacturability, expression levels, downstream processing and formulation conditions required for stable long term storage. (54, 55). During the efforts to produce anti-Ebola antibodies in CHO cells, 13C6FR1 proved to be particularly challenging with respect to cell culture and downstream processing. We interrogated the behavior of two parental chimeric 13C6 versions. The two parental 13C6 versions, 13C6FR1 and 13C6mu, consisted of identical sequences except for framework 1 of the variable light (VL) and variable heavy (VH) domains. Framework 1 of 13C6FR1 VL contained 4 amino acid differences and the 13C6FR1 VH framework 1 contained 15 differences as compared to 13C6mu. 13C6FR1 reflected the optimization by Mapp Biopharmaceuticals for expression in tobacco, whereas 13C6mu was the original mouse hybridoma sequence. Sequence analysis indicated many opportunities for engineered optimization for CHO cell expression as well as potential improvements to antibody stability. (55). However, time and resource constraints did not allow for the generation of hundreds of variants and subsequent binding and activity testing to identify improved antibodies. Therefore, only one modification was made to the C-terminal end of the VL in both parental versions as the composition was highly unusual. (56). Human germline analysis indicated a conserved lysine was missing at position 148 (based on the AHo numbering scheme). (57). The missing K148 shortens the linker length between the variable and constant regions and could potentially alter the surface charge properties of the antibody. It was hypothesized that alterations of both parental versions could have an effect on stability and expression. Therefore, one variant was designed for each 13C6FR1 and 13C6mu with a lysine in position 148 and an arginine in position 149 of the VL. Although these modifications were distal to the complementary determining regions (CDRs), it was noted the paratope structure could still be altered and hence, potentially affect bioactivity. (58), This study evaluated all four antibodies during cell culture and purification to assess the effect of sequence composition on manufacturability and product quality. Understanding the effect of sequence composition could result in a molecule that is more manufacturable and would enable a quicker response to future epidemics.

### Results

### 13C6 Light Chain Variant

Both the parental 13C6FR1 and 13C6mu light chains have non-standard germline composition on the C-terminal end of the VL domain. The germline contains a lysine at position 148 and arginine at position 149 whereas the parental 13C6 contains an arginine at position 148 and nothing at position 149. The unusual parental composition in this region could have an impact on IgG stability, VL-VH interface interaction and expression titer. (59). Therefore, position 148 was substituted with a lysine and an arginine was inserted at position 149 for both 13C6FR1_LC and 13C6mu_LC. The variants were named ‘13C6FR1+K’ and ‘13C6mu+K’ due to the R148K substitution. This nomenclature will be used throughout this manuscript.

Bioreactor production of the 13C6 Variants in CHO Cells.

To evaluate the expression and product quality of the 13C6 variants, Amgen's CHO DXB-11 clonal host cell line was transfected with Amgen expression vectors. Four transfectants of each antibody were selected for growth in media lacking glycine, hypoxanthine and thymidine (−GHT media). Once viability of the −GHT selected pools reached greater than 85%, a 24-deep well plate production assay was performed to identify the highest expressing pool of each antibody. Due to time constraints, pools were not amplified with methotrexate and only the top expressing −GHT pool of each antibody was carried forward into bioreactors (data not shown). Two liter (2 L) perfusion bioreactors were run in duplicate for 14 days to assess antibody titers and provide material for downstream processing. To ensure the −GHT pools did not overgrow, a temperature shift was implemented to control cell growth. Temperature shifts were based on cell density and did not occur on the same day for all the bioreactors. The 13C6FR1 parent and 13C6FR1+K cultures were temperature shifted to 32.5° C. on day 6, one day earlier than the 13C6mu and 13C6mu+K bioreactors. Despite an initial temperature shift to 32.5° C., continued growth was observed, therefore all of the bioreactors were temperature shifted again on day 9 to 31.5° C. to further suppress growth. Antibody production of each pool was monitored over the days of culture. The titer results showed higher antibody production with the lysine insertion for both the 13C6FR1+K and 13C6mu+K variants (Table 3). All antibody sequences were verified by mass spectroscopy with 100% sequence coverage.

TABLE 3
Day 14 titer and protein A pool HMW content for 13C6 variants

| | Day 14 | |
|---|---|---|
| | titer | HMW |
| Construct | (g/L) | (%) |
| 13C6mu | 4.23 | 8.85 |
| 13C6mu + K | 5.79 | 7.20 |
| 13C6FR1 | 3.05 | 25.20 |
| 13C6FR1 + K | 5.13 | 6.25 |

Downstream Processing.

Purification of the four antibodies was performed to supply material for analytical characterization. In addition, the effect of the sequence modifications on downstream operations and general manufacturability were evaluated. Harvest for each of the antibodies was performed by centrifugation on day 14 of the cell culture production. Antibody capture from the harvested cell culture fluid (HCCF) was achieved by protein A chromatography. Polishing chromatography was performed by either HIC or CEX and the resulting pools were formulated to 20 mM acetate, 250 mM sucrose, pH 5. Typical performance was observed during protein A chromatography with all the antibodies. The most notable differences between the antibodies were slight differences in elution pool turbidity and the HMW content of the protein A elution pool (Table 3).

The HMW content ranged from 6.25% through 25.2%. 13C6FR1 had exceptionally high HMW, averaging 25.2%. The high HMW burden for 13C6FR1 would likely require multiple chromatography steps for aggregate reduction and result in an overall low yield. When comparing 13C6FR1 and 13C6FR1+K, the lysine insertion significantly reduced the HMW content in the protein A pool (average of 6.25% for 13C6FR1+K). 13C6mu and 13C6mu+K had similar levels of HMW (7.2%-8.85%). It should be noted that these are unamplified pools; clones producing lower HMW levels could potentially be selected during cell line development.

Cation exchange chromatography (CEX) is a common unit operation to reduce HMW in mAb processes. (60,61). The 13C6 variants were evaluated on the strong cation exchanger Fractogel SO3-. Fractions (0.5CV) were collected over the elution peak and analyzed for protein concentration and HMW. A comparison of the purity plots show that under the same chromatography conditions, higher purity was achieved at higher yield for the 13C6 antibodies with the lysine insertion (13C6FR1+K and 13C6mu+K). In contrast, the parental sequences (13C6FR1 and 13C6mu) had lower purity and recovery. It should be noted that the parental sequences both had higher starting HMW in the protein A pool compared to the lysine insertion variants. It is also likely that additional resin screening could be performed or the CEX operating conditions could be optimized to further improve yield and purity for each individual antibody.

Hydrophobic interaction chromatography (HIC) is also a common aggregate polishing step for mAb products (60-62), therefore, the effect of the sequence changes on HIC performance was examined. When comparing the cumulative purity versus the cumulative yield, there was not a dramatic difference between 13C6mu and 13C6mu+K. In contrast, there was a significant difference between 13C6FR1 and 13C6FR1+K. Higher step yield and overall purity was achieved for 13C6FR1+K. HMW was observed in the early fractions for 13C6FR1, which would also further decrease the step yield if the BMW was removed. The HIC conditions tested were not designed for each specific molecule and it is likely that the HIC conditions could be optimized to improve yield and purity for each antibody.

In addition to step yield and chromatographic performance, the solution stability of the 13C6 variants was examined over a range of conditions that are relevant for downstream processing. In these experiments, the pH and salt strength of the protein A pool were varied from pH 5-pH 7.5 and 0 mM-200 mM NaCl by the addition of stock solutions. Light scattering measurements were taken at 405 nm in a microtiter plate on a Tecan plate reader after 24 hours. The highest turbidity was observed with 13C6FR1. For 13C6FR1, as the pH increased and the salt concentration decreased, there was an increase in turbidity. The highest turbidity observed for the 13C6FR1 variant was approximately 0.2 AU at ≥pH 6 and ≤50 mM sodium chloride. While, in this system, turbidity has not been directly correlated to filter capacity, this level of precipitation could create column backpressure and/or filtration challenges in a manufacturing setting. As a result of the precipitation, there is also a smaller operating space for 13C6FR1 which could limit purification options (anion-exchange chromatography, AEX for example). In contrast, 13C6FR1+K had A405 values≤0.075 AU across the entire range tested. 13C6mu and 13C6mu+K both had A405 values≤0.125 AU and there did not appear to be a difference between the two antibodies.

The effect of pH and ionic strength on aggregation was also evaluated with the 13C6 antibodies. In this study, the pH and salt strength of the protein A pool was varied from pH 5.0-7.5 and 0-200 mM NaCl by the addition of stock solutions. Samples were analyzed for HMW by size exclusion chromatography (samples were analyzed within 24 hours of preparation). The highest HMW was observed with 13C6FR1. For 13C6FR1, the aggregate content was strongly influenced by the pH. As the pH increased there was an increase in HMW, with an overall range of 11.6% through 19.7%. There was only a minor effect of salt strength on 13C6FR1 HMW levels. The sensitivity to pH (and NaCl concentration) was eliminated with the lysine insertion (13C6FR1+K). 13C6mu and 13C6mu+K were not sensitive to pH or NaCl concentration within the ranges tested. Aside from the absolute aggregate level, the most noticeable difference observed between the different sequences was the sensitivity to operating conditions. The 13C6 FR1+K, 13C61mu, and 13C6mu+K variants had freedom to operate over a wide range of pH and salt strength, meaning that there would be few limits on the downstream processing options relative to solution stability. In contrast, 13C6FR1 had a dramatic increase in HMW when the pH was increased, which would limit the downstream operating space.

The thermal stability of the different 13C6 variants in downstream relevant buffer systems was measured by a high throughput extrinsic fluorescence assay using SYPRO orange.²³ The thermal transition temperatures measured by this method are shown in Table 4. Overall, 13C6FR1 had the lowest unfolding temperature in the downstream buffer conditions at ≤60.8° C. under all conditions tested. The lysine insertion sequence (13C6FR1+K) had a significantly higher unfolding temperature than 13C6FR1 for all buffer systems screened. Although higher than 13C6FR1, 13C6mu also had a fairly low unfolding temperature, which improved with the lysine insertion (13C6mu+K). There were no obvious trends for the parental sequences (13C6FR1 and 13C6mu) with respect to solution conditions. In contrast, the lysine insertion sequences (13C6FR1+K and 13C6mu+K) both showed an increase in unfolding temperature as the solution pH increased (in downstream buffers). There was also a decrease in unfolding temperature as the NaCl concentrations increased; however, the difference was small. The relative differences in unfolding temperature can be indicative of solution stability (such as aggregation propensity in formulation) and susceptibility to stresses during downstream processing, such as surface mediated unfolding or solution dependent aggregation. (63-66).

TABLE 4
Unfolding temperature (° C.) as
a function of pH and NaCl concentration

| Construct | NaCl (mM) | pH 5 | pH 6 | pH 7.5 |
|---|---|---|---|---|
| 13C6FR1 | 0 | 60.8 | 60.6 | 58.6 |
| | 50 | 59.2 | 59.6 | 59.0 |
| | 200 | 58.6 | 59.8 | 59.2 |
| 13C6FR1 + K | 0 | 64.6 | 67.4 | 69.0 |
| | 50 | 63.4 | 66.6 | 68.8 |
| | 200 | 62.8 | 65.6 | 68.4 |
| 13C6mu | 0 | 62.2 | 62.6 | 61.6 |
| | 50 | 61.0 | 61.8 | 61.4 |
| | 200 | 61.0 | 61.8 | 61.6 |
| 13C6mu + K | 0 | 64.8 | 67.4 | 69.0 |
| | 50 | 63.8 | 66.4 | 68.6 |
| | 200 | 62.2 | 66.0 | 68.4 |

Analytics/Biophysical Characterization.

To better understand the differences in thermal stability, each 13C6 antibody was analyzed by differential scanning calorimetry (DSC) to evaluate thermal transition temperature (Tₘ) of the individual antibody domains (CH2, CH3, and Fab domains). The resulting DSC thermograms for 13C6FR1 and 13C6mu displayed a profile that suggested the Fab domain was the first domain to unfold with transition temperatures of approximately 66° C. and 68° C. respectively (Table 5). 13C6FR1+K and 13C6mu+K showed a significant shift in the thermogram profiles in contrast to 13C6FR1 and 13C6mu. The addition of the lysine to both constructs resulted in an increase in the thermal stability of the Fab domain. The Tₘ for the Fab domain for 13C6FR1+K and 13C6mu+K was between 8° C. and 10° C. higher than 13C6FR1 and 13C6mu. As a result of the increased Fab domain stability, the first domain unfolding event shifted from the Fab to the CH2 domain. Furthermore, the addition of lysine resulted in highly similar DSC profiles for 13C6FR1+K and 13C6mu+K. The Tₘ for the CH2 and CH3 domains were similar for all of the 13C6 antibodies.

TABLE 5
DSC Average Thermal Transition Temperatures

| | Tm (° C.) | | |
|---|---|---|---|
| Sample Name | Fab | CH2 | CH3 |
| 13C6FR1 | 66.0 | 71.2 | 83.8 |
| 13C6FR1 + K | 76.5 | 71.4 | 84.5 |
| 13C6Mu | 68.2 | 72.8 | 83.7 |
| 13C6Mu + K | 76.5 | 71.7 | 84.3 |

The Ebola Zaire binding ELISA was used to assess the binding activity of the CHO produced 13C6 antibodies. Full dose response curves for the binding activity for the CHO produced 13C6 antibodies were compared to the binding activity of the tobacco produced 13C6FR1 antibody currently used in ZMapp™ (tobacco c13C6FR1). The percent relative potency was calculated as a ratio of the EC₅₀ values for the tobacco 13C6FR1 to the test sample (Table 6). 13C6FR1 compared to tobacco c13C6FR1 had a relative potency of 100%, demonstrating that the change in the expression system (tobacco vs CHO) had no impact on the binding of the molecule in the assay. 13C6FR1+K showed a higher or increased percent relative potency when compared to tobacco c13C6FR1 (121%).

TABLE 6
% Relative Potency for 13C6 antibodies

| | % Relative |
|---|---|
| Sample Name | Potency |
| 13C6FR1 | 100 |
| 13C6FR1 + K | 121 |
| 13C6Mu | 55 |
| 13C6Mu + K | 73 |

13C6mu with and without the lysine insertion had lower percent relative potency compared to the tobacco 13C6FRI: 55% for 13C6mu and 73% for 13C6mu+K (Table 6). The reduced percent relative potency is likely due to the significant sequence differences in the Fv region between the FR1 and mu variants. However, the addition of the lysine appears to increase the binding of the mu construct but does not equate to equivalent binding to the 13C6FR1 CHO material.

In Silico Modeling.

To understand the potential mechanism of differential aggregation formation between the antibodies, in silica homology models were constructed for each Fab and their exposed hydrophobicity was compared using two methods. The Spatial Aggregation Propensity algorithm (67) revealed a motif that was intense in 13C6FR1 but was less intense with the lysine 148 insertion. This predicted aggregation prone region might be sufficient to have induced aggregation formation of 13C6FR1 whereas the phenomena was remediated in the presence of lysine 148 and/or arginine 149. Both 13C6mu and 13C6mu+K exhibited similar aggregation behavior whether K148 and R149 was present or absent. This is probably explained by the lack of the 13C6FR1 predicted aggregation prone region. The different VL residue content in framework 1 between the murine and the tobacco-optimized 13C6FR1 manifests as the lack of the intense aggregation prone region revealed in 13C6FR1. The residue differences in framework 1 of the VL were revealed to be within the predicted aggregation prone region.

Discussion

In the early months of 2014 the largest outbreak of Ebola, so far, resulted in more deaths from this epidemic than all previous Ebola outbreaks combined. Between March and October there were nearly 10 thousand infections and thousands of confirmed deaths, and by late fall of that year, the trajectory of new infections in West Africa and globally was uncertain.

At that time, the worldwide focus on solutions was broad-based and included the development of new therapies such as the ZMapp™ antibody cocktail, which had displayed promising pre-clinical results, but was not available in sufficient quantity to address the scope of this epidemic. In order to have a meaningful impact on this large scale Ebola outbreak, a consortium of non-profit, governmental, and industrial partners was formed to deliver sufficient anti-Ebola mAbs derived from CHO cells as rapidly as possible. The objectives of the consortium were to produce thousands of doses of anti-Ebola mAbs, with product attributes as comparable as possible to tobacco derived ZMapp™ and initiate a clinical study with patients in need by Jun. 1, 2015.

Fortunately, the Ebola outbreak subsided in early 2015 and the need for large quantities of clinical doses diminished. Through the efforts of the consortium we have made technical advances in the processing of CHO derived anti-Ebola antibodies, specifically for 13C6FR1, that may be beneficial in the event of a future Ebola virus outbreak or other pandemic viral outbreaks where mAbs are considered potential therapeutics.

This study has demonstrated that the sequence of a monoclonal antibody can significantly impact both expression and biophysical behavior, which can strongly affect manufacturability. For 13C6FR1, the insertion of a single lysine at position 148 effected aggregation, reducing HMW levels from 25% to 6%, which was similar to the HMW levels of I3C6mu and 13C6mu+K. Decreased aggregation propensity of 13C6FR1+K as compared to 13C6FR1 could be due to a structural change and improvement in thermal stability, as evidenced by the significant increase in the Tm for the Fab domain of 13C6FR1+K. The presence of additional electropositive charge from arginine 149 could also have contributed to the observed reduction in aggregate formation.

Studies were also performed to assess the impact of sequence modification on upstream and downstream operations. From an upstream perspective, antibody titers were higher in the presence of the lysine insertion in these unamplified pools. Expression levels will most likely increase upon methotrexate amplification and after clone screening. However, due to time constraints, these experiments were not performed and therefore it is unknown if the same titer differences will be found after more intensive cell line development.

In multiple downstream conditions it was found that 13C6FR1 was challenging due to higher initial aggregate levels as well as a higher propensity to aggregate and precipitate. As demonstrated by these data, 13C6FR1 had the most limited operating space with regard to pH and NaCl concentrations. For 13C6FR1, higher operational pH resulted in elevated HMW. Additionally, at elevated pH and lower conductivity there was an increase in product precipitation. The ultimate effect of the observed solution instability would be to limit the available operational space for 13C6FR1. For example, with 13C6FR1 operation at greater than pH 5.5 would cause unacceptable increases in HMW and turbidity and could result in heavy yield losses to meet drug substance (DS) product quality targets.

Insertion of the lysine in the light chain resulted in lower initial aggregate levels and decreased the susceptibility to aggregation and precipitation under downstream conditions. Additionally, the lysine insertion had a positive effect on step yield for 13C6FR1. At similar purity, 13C6FR1+K had a 27.6% improvement in step yield over 13C6FR1 during CEX chromatography and an 8.6% improvement in step yield during HIC chromatography. Overall, the improved downstream behavior of 13C6FR1+K resulted in lower DS HMW, higher yield, and a wider potential downstream operating space.

While this study demonstrated no negative impact to in vitro binding as measured by ELISA, data suggests 13C6 may act through effector function binding. (28). Cell based potency and other effector function assays were not employed here. Mouse studies are currently being conducted to better understand the potential impact of sequence optimization on in vivo efficacy.

In the process of working through the issues of rapidly producing anti-Ebola antibodies there were several additional technical and logistical lessons learned that should be shared:

(1) Given the high viral load of the Ebola virus among infected patients the dose is relatively high at approximately 10 grams of antibody cocktail per cycle. For the treatment of thousands of patients, perhaps as much as 100 kg of cocktail antibodies may be needed which could require several months of production in a multi-2000L CHO-cell bioreactor facility. Engineering sequences to improve expression and purification yield is a powerful tool in reducing mass requirements through manufacturing.

(2) While achievable, adequate response to future threats will require additional technological innovation. As demonstrated in this manuscript, the single most impactful improvement that can be made to the overall development process is to design a biotherapeutic with the optimal sequence. The benefit of a single lysine insertion into the light chain of 13C6FR1 was demonstrated in this study; however, many other sequence alterations could be made to potential hot spots in order to improve stability, and expression.

(3) The cycle time from outbreak to resolution of an epidemic can be short (many months in duration) relative to typical mAb therapeutic development timelines (many years in duration) and therefore a “rapid response” approach is critical. It is not always feasible to re-engineer antibody sequences, transfect new cell lines, establish reliable production capacity, bridge results from clinical trial information, or establish product comparability between innovator and modified product in the time frame required for a rapid response. As much as possible, these critical issues need to be addressed in advance in order to reduce the impact of a viral outbreak such as EVD.

(4) The regulatory pathways for compassionate use, in the case of a worldwide threat, should be dramatically streamlined and well known. Specifically, this consortium contemplated producing GMP batches with unamplified pools rather than amplified clones. To challenge conventional regulatory pathways, product quality from unamplified pool mAb production should be generated to demonstrate lot-to-lot consistency. These data could shift the current regulatory agency paradigm, at least for cell lines where this data can be established, thus enabling a more rapid response time in the event of a future Ebola type outbreak.

(5) The commercial institutions that participated in this consortium should be commended for their participation as they did so with the knowledge that they could leverage their capabilities and available capacity to help in some way on behalf of the patients infected with EVD, but also with some risk to their ongoing corporate obligations. However, future response to viral outbreaks should not depend on the goodwill of corporate entities, whose capabilities and capacity may change over time. To reduce the impact of future outbreaks, a global strategy needs to be put in place that focuses on the development of technologies that enable improved molecules with intensified processes, and provides access to manufacturing capacity, experienced operations staff, cell lines, and raw materials that can provide a robust and rapid response.

### Materials and Methods

### Material

Tobacco produced 13C6FR1 (control), CHO produced 13C6 FR1+/−K insertion, CHO produced 13C6 mu+/−K insertion.

### Molecular Biology

Protein sequences for antibodies 13C6FR1 and 13C6mu were provided by Mapp Biopharmaceuticals (San Diego, Calif.). The sequences were back-translated into codons that were optimized for mammalian expression using VectorNTI (Life Technologies, Carlsbad, Calif.). The antibody coding DNA along with the VK1|012 signal peptide were synthesized by Integrated DNA Technologies (Coralville, Iowa) and ligated into Amgen expression vectors. The light chain variants were generated using the Quikchange site-directed mutagenesis kit (Stratagene, La Jolla, Calif.). Both strands of the entire plasmid for each of the 6 DNA constructs were sequence confirmed using BigDye Terminator sequencing (life Technoogies, Carlsbad, Calif.).

### Transfections

Amgen's CHO DXB11 host cell line was transfected with Amgen expression vectors containing 13C6 constructs. DNA was linearized with Pvu1, followed by ethanol precipitation. Four pools of each condition were transfected in 96-well plates (Bio-Rad Gene Pulser MXcell Electroporation System) following the high DNA content high-throughput electroporation method. (68). Cells were selected in GHT depleted media. Once viability of the pools reached >85%, cells were seeded into a 24-deep well plate production assay. All four pools of both 13C6FR1 and 13C6FR1+K were seeded into production in triplicate, whereas 13C6mu constructs took slightly longer to recover from −GHT selection and did not have as many replicates. Productions were seeded at 10×10⁶ c/mL×2 mLs of Amgen cell culture medium in 24-deep well plates at 36° C., 5% CO₂, shaking at 225 rpms. On days 1-3, plates were spun down at 1000 rpm for 5 minutes, 70% of the medium was removed and replaced with Amgen medium. Samples were harvested on day 4 and analyzed for titer. The highest producing pool titer from each condition was selected and scaled-up for bioreactors.

### Bioreactor Experiments

Minus GHT pools from each construct were grown in shake flasks in 5% CO₂, humidified Kuhner (Basel, Switzerland) incubators for the N-3 and N-2 stages. Cells were subsequently seeded into 3 L stirred-tank bioreactors for the N-1 stage and N (production). Chemically defined medium with no animal derived components was used throughout the growth phase and production. pH and dissolved oxygen setpoints were maintained throughout the course of production. pH was controlled with 1.0 M sodium carbonate and carbon dioxide gas. Temperature setpoint was lowered to control cell growth as cells reached high cell densities. Ex-Cell™ antifoam (SAFC) was used as needed to control foaming.

Viable cell density (VCD) and viability were determined using a ViCell XR automated cell counter (Beckman Coulter, Brea, Calif.). The osmolality was determined using a Model 2020 osmometer (Advanced Instruments, Norwood, Mass.). pH and CO2 readings were determined using a Rapidlab 1260 Blood Gas Analyzer (Siemens, Malvern, Pa.). Titer was determined using affinity protein A ultra-high performance liquid chromatography (Waters, Milford, Mass.). Glucose and lactate were measured using a Bioprofile Flex (Nova Biomedical, Waltham, Ma.). All 3 L bioreactors were manufactured by Applikon (Delft, Netherlands). Supernatant was harvested on day 14 by centrifugation for 30 minutes at 3000 rpm, and filtered through a Pall Acropak 500 capsule)

### Sequence Confirmation

The sequence of each antibody was confirmed using mass spectrometry. (69).

### Size Exclusion Chromatography (SEC) Analysis of Aggregation

60 μg of each sample was separated isocratically at ambient temperature (˜22° C.) using a sodium phosphate mobile phase (100 mM sodium phosphate, 250 mM sodium chloride, pH 6.8) on a 4.6×150 mm, 1.7 μm Waters Acquity BEH200 SEC column. The column flow rate was 0.4 mL/min and detection was monitored by A280 with a method run time of 6 minutes.

### High Throughput Screening

Downstream HTS studies were performed using a Tecan Freedom EVO™ robotic liquid handling system (Tecan US, Research Triangle Park, N.C., USA). The Tecan was configured with Te-Chrom components to facilitate automated micro-chromatography. Measurements for concentration and turbidity were performed on an integrated microplate reader. Resins were acquired as pre-packed 450 μL Robocolumns from Atoll (Weingarten, Germany). Product pools were collected as fractions (1 CV/fraction) in 96-deep well plates (Qiagen Sciences, Germantown, Md., USA) for further analysis. Results from individual fractions were used to generate pseudo-chromatograms. Cumulative results from each fraction were used to calculate yield and mass balance.

### Column Chromatography

Bench scale chromatography experiments were conducted using an Akta Explorer 100 (GE Healthcare, Piscataway, N.J., USA). Resins were packed into 1.15 cm ID Vantage columns (EMD Millipore, Billerica, Mass., USA) to a bed height of approximately 10-25 cm). The resins Mab Select SuRe™ and Phenyl Sepharose 6 Fast Flow hi sub were acquired from GE Healthcare (Piscataway, N.J., USA) and Fractogel SO3-was acquired from EMD Millipore (Billerica, Mass., USA). Protein A chromatography was used as initial capture and purification from harvested cell culture fluid (HCCF). The second chromatography step was either cation exchange chromatography (Fractogel SO3-) performed in BEM or hydrophobic interaction chromatography (Phenyl Sepharose 6 Fast Flow hi sub) performed in FTM. For CEX, following equilibration, the column was loaded with neutralized protein A pool, washed with equilibration buffer, and eluted using a NaCl linear gradient over 10 column volumes. For HIC, the protein A pools were conditioned to the target loading conditions and then applied to the column and followed by a 6 column volume (CV) wash with equilibration buffer. The load flow through and wash volumes were collected as the process pool. Purified product pools were formulated to 20 mM acetate, 250 mM sucrose pH 5 using a 30 kD regenerated cellulose Tangential Flow Filtration membrane (EMD Millipore, Billerica, Mass., USA). Polysorbate 80 was added to the final product pool to a concentration of 0.01% (v/v). (63).

### Potency

A proprietary Ebola glycoprotein (GP) binding ELISA assay was provided to Amgen by MappBio to measure potency. The ELISA is designed to monitor the dose-dependent binding of anti-Ebola antibodies to the Ebola Zaire glycoprotein. The glycoprotein is coated onto a 96-well polystyrene microtiter assay plate. After coating, the plate is blocked with reagent to prevent non-specific binding of the anti-Ebola antibodies. A dilution series of the reference material (RS) and test samples are then added. The amount of bound anti-Ebola antibody is detected by an anti-human kappa horseradish peroxidase (HRP) conjugated detection antibody, followed by the chromogenic substrate tertramethyl benzidine (TMB). The percent activity of each test sample is calculated from the EC₅₀ values of the test sample and the reference standard dose-response curves.

### Differential Scanning Calorimetry (DSC)

Differential Scanning calorimetry (DSC) analysis was performed using a Capillary VP-DSC (MicroCal, Northampton, Mass.) with a scan rate of 1° C./min and no feedback. Immediately prior to analysis, all samples were diluted to approximately 0.5 mg/mL in 20 mM acetate, 250 mM sucrose, 0.01% polysorbate 80 pH 5.0. The DSC profiles were analyzed using MicroCal Origin v 7.0 software (MicroCal, Northampton, Mass.). All theromograms were baseline corrected and normalized to the moles of protein loaded. The Tₘ for each sample was determined using the MicroCal Origin Non-2 State fitting algorithm with 3 peaks.

### In Silico Modeling

Fv models for 13C6FR1 and 13C6mu were made using the the Antibody Modeler in the Molecular Operating Environment (MOE, Chemical Computing Group, Montreal CA). The Amber:EHT 10 force field was used with GB/VI ranking to arrive at the working Fv model. Non-ideality (non-proline cis-peptides, atom clashes, etc.) in the model was corrected manually. Low-mode molecular dynamics was performed on the CDR3 VH loop and any non-ideality (described above) corrected. Constant light and CH1 domains were then ligated to the Fv models. The domain linkers were energy minimized followed by solid-body minimization to allow the four domains to arrive at a low-energy relationship. The lysine 148 insertions were then performed (resulting in shifting R148 to become R149) on these Fab models. The linkers were energy minimized followed by solid-body energy minimization as before.

The MOE Patch Analyzer was employed to calculate exposed electropositive, electronegative and hydrophobic surfaces. Patch size, number, composition and location were compared between each of the four antibodies.

The Spatial Aggregation Propensity algorithm within Discovery Studio 4.1 (Biovia, San Diego, Calif.) was used to calculate potential aggregation prone regions for each of the four antibodies. Each Fab was loaded into Discovery Studio 4.1 and Prepare Protein performed with the CHARMm force field applied. The Cutoff Radius parameter was set to either 5 Å or 10 Å and all other settings were kept at default.

### Example 2: Selection of the Best mAb Combinations

### Materials and Methods

### Ethics Statement

The guinea pig experiment, in addition to the second and third NHP study (ZMapp1, ZMapp2 and ZMAPP) were performed at the National Microbiology Laboratory (NML) as described on Animal use document (AUD) #H-13-003, and has been approved by the Animal Care Committee (ACC) at the Canadian Science Center for Human and Animal Health (CSCHAH), in accordance with the guidelines outlined by the Canadian Council on Animal Care (CCAC). The first study with MB-003 in NHPs was performed at United States Army Medical Research Institute of Infectious Diseases (USAMRIID) under an Institutional Animal Care and Use Committee (IACUC) approved protocol in compliance with the Animal Welfare Act, Public Health Service Policy, and other federal statutes and regulations relating to animals and experiments involving animals. The facility where this research was conducted in accredited by The Association for Assessment and Accreditation of Laboratory Animal Care International and adheres to principles stated in the 8^{th} edition of the Guide for the Care and Use of Laboratory Animals, National Research Council 2011.

### mAb Production

The large-scale production of mAb cocktails cZMAb, MB-003, ZMapp1, ZMapp2 and ZMAPP in addition to control mAb 4E10 (anti-HIV) from N. benthamiana under GMP conditions was done by Kentucky BioProcessing (Owensboro, Ky.) as described previously (28) (30) (33). The large-scale production of m4G7 was performed by the Biotechnology Research Institute (Montreal, QC) using a previously described protocol (31).

### Viruses

The challenge virus used in NHPs was Ebola virus H. sapiens-tc/COD/1995/Kikwit-9510621 (EBOV-K) (order Mononegavirales, family Filoviridae, species Zaire ebolavirus; GenBank accession #AY354458)(34). Passage three from the original stock was used for the studies at the NML and passage four was used for the study performed at USAMRIID (the NHP study with the individual MB-003 mAbs). Sequencing of 112 clones from the passage three stock virus revealed that the population ratio of 7U:8U in the EBOV GP editing site was 80:20; sequencing for the passage four stock virus was not performed, and therefore the ratio of 7U:8U in the editing site was unknown. The virus used in guinea pig studies was guinea pig-adapted EBOV, Ebola virus VECTOR/C. porcellus-lab/COD/1976/Mayinga-GPA (EBOV-M-GPA) (order Mononegavirales, family Filoviridae, species Zaire ebolavirus; Genbank accession number AF272001.1) (35). The Guinean variant used in IgG ELISA and neutralizing antibody assays was Ebola virus H. sapiens-tc/GIN/2014/Gueckedou-C05 (EBOV-G) (order Mononegavirales, family Filoviridae, species Zaire ebolavirus; GenBank accession #KJ660348.1) (17).

### Animals

Outbred 6-8 week old female Hartley strain guinea pigs (Charles River) were used for these studies. Animals were infected IP with 1000×LD50 of EBOV-M-GPA. The animals were then treated with one dose of ZMAb, MB-003, ZMapp1, ZMapp2, c13C6, h13F6 or c6D8 totaling 5 mg per guinea pig, and monitored every day for 28 days for survival, weight and clinical symptoms. This study was not blinded, and no animals were excluded from the analysis.

For the MB-003 study performed at USAMRIID, thirteen rhesus macaques (Macaca mulatta) were obtained from the USAMRIID primate holding facility, ranging from 5.1 to 10 kg. This study was not blinded, and no animals were excluded from the analysis. Animals were given standard monkey chow, primate treats, fruits, and vegetables for the duration of the study. All animals were challenged IM with a target dose of 1000 PFU. Treatment with either monoclonal antibody, MB-003 cocktail, or PBS was administered on 1, 4, and 7 dpi via saphenous intravenous infusion. Animals were monitored at least once daily for changes in health, diet, behavior, and appearance. Animals were sampled for chemical analysis, complete bloods counts and viremia on 0, 3, 5, 7, 10, 14, 21, and 28 dpi.

For the ZMapp1 and ZMapp2 study, fourteen male and female rhesus macaques (Macaca mulatta), ranging from 4.1 to 9.6 kg (4-8 years old) were purchased from Primgen (USA). This study was not blinded, and no animals were excluded from the analysis. Animals were assigned groups based on gender and weight. Animals were fed standard monkey chow, fruits, vegetables, and treats. Husbandry enrichment consisted of visual stimulation and commercial toys. All animals were challenged IM with a high dose of EBOV [backtiter: 4000×TCID₅₀ or 2512 PFU] at 0 dpi. Administration of the first treatment dose was initiated at 3 dpi, with identical doses at 6 and 9 dpi. Animals were scored daily for signs of disease, in addition to changes in food and water consumption. On designated treatment days in addition to 14, 21, and 27 dpi, the rectal temperature and clinical score were measured, and the following were sampled: blood for serum biochemistry and cell counts and viremia. This study was not blinded, and no animals were excluded from the analysis.

For the ZMAPP study, twenty-one male rhesus macaques, ranging from 2.5 to 3.5 kg (2 years-old) were purchased from Primgen (USA). This study was not blinded, and no animals were excluded from the analysis. Animals were assigned groups based on gender and weight. Animals were fed standard monkey chow, fruits, vegetables, and treats. Husbandry enrichment consisted of visual stimulation and commercial toys. All animals were challenged IM with EBOV [backtiter: 1000×TCID₅₀ or 628 PFU] at 0 dpi. Administration of the first treatment dose was initiated at 3, 4 or 5 dpi, with two additional identical doses spaced three days apart. Animals were scored daily for signs of disease, in addition to changes in food and water consumption. On designated treatment days in addition to 14, 21, and 28 dpi, the rectal temperature and clinical score were measured, and the following were sampled: blood for serum biochemistry and cell counts and viremia.

### Blood Counts and Blood Biochemistry

Complete blood counts were performed with the VetScan HM5 (Abaxis Veterinary Diagnostics). The following parameters were tested: levels of white blood cells (WBC), lymphocytes (LYM), percentage of lymphocytes (LYM %), levels of platelets (PLT), neutrophils (NEU) and percentage of neutrophils (NEU %). Blood biochemistry was performed with the VetScan VS2 (Abaxis Veterinary Diagnostics). The following parameters were tested: levels of alkaline phosphatase (ALP), alanine aminotransferase (ALT), blood urea nitrogen (BUN), creatinine (CRE), and total bilirubin (TBIL).

### Enzyme-Linked Immunosorbent Assays (ELISAs)

IgG ELISA with c13C6, c2G4 or c1H3 was performed as described previously (31) using gamma-irradiated EBOV-G and EBOV-K virions purified on a 20% sucrose cushion as the capture antigen in the ELISA. Each mAb was assayed in triplicate.

### Neutralizing Antibody Assays

Two-fold dilutions of c13C6, c2G4 or c1H3 ranging from 0.0156 to 2 mg were first incubated with 100 PFU of EBOV-G at room temperature for 1 hour with or without complement, transferred to Vero E6 cells and incubated at 37° C. for 1 hour, and then replaced with DMEM supplemented with 2% fetal bovine serum and scored for the presence of cytopathic effect (CPE) at 14 dpi. The lowest concentrations of mAbs demonstrating the absence of CPE were averaged and reported.

### EBOV Titration by TCID₅₀ and RT-qPCR

Titration of live EBOV was determined by adding 10-fold serial dilutions of whole blood to VeroE6 cells, with three replicates per dilution. The plates were scored for cytopathic effect at 14 dpi, and titers were calculated with the Reed and Muench method (36). Results were shown as median tissue culture infectious dose (TCID₅₀).

For titers measured by RT-qPCR, total RNA was extracted from whole blood with the QIAmp Viral RNA Mini Kit (Qiagen). EBOV was detected with the LightCycler 480 RNA Master Hydrolysis Probes (Roche) kit, with the RNA polymerase (nucleotides 16472 to 16538, AF086833) as the target gene. The reaction conditions were as follows: 63° C. for 3 min, 95° C. for 30 s, and cycling of 95° C. for 15 s, 60° C. for 30 s for 45 cycles on the ABI StepOnePlus. The lower detection limit for this assay is 86 genome equivalents/ml. The sequences of primers used were as follows: EBOVLF2 (CAGCCAGCAATTTCTTCCAT), EBOVLR2 (TTTCGGTTGCTGTTTCTGTG), and EBOVLP2FAM (FAM-ATCATTGGCGTACTGGAGGAGCAG-BHQ1).

### Sequence Alignment

Protein sequences for EBOV-K and EBOV-G surface glycoproteins were obtained from GenBank, accession numbers AGB56794.1 and AHX24667.1 respectively. The sequences were aligned using DNASTAR Lasergene 10 MEGAlign using the Clustal W algorithm.

### Statistical Analysis

For the guinea pig and nonhuman primate studies, each treatment group consisted of six animals. Assuming a significance threshold of 0.05, a sample size of six per group will give >80% power to detect a difference in survival proportions between the treatment (83% survival or higher) and the control group using a one-tailed Fisher's exact test.

Survival was compared using the log-rank test in GraphPad PRISM 5, differences in survival were considered significant when the p-value was less than 0.05. Antibody binding to EBOV-G and EBOV-K was compared by fitting the data to a 4-parameter logistic regression using GraphPad PRISM 5. The EC₅₀ were considered different if the 95% Confidence Intervals excluded each other. For all statistical analyses, the data conformed to the assumptions of the test used.

### Selection of the Best mAb Combinations

The c13C6 mAb of the following experiments is comprised of light chain variant disclosed in SEQ ID NO: 24 and the heavy chain variant disclosed in SEQ ID NO: 23. The constant regions are human (IgG1-Kappa).

Our efforts to down-select for an improved mAb cocktail comprising components of MB-003 and ZMAb began with the testing of individual MB-003 antibodies in guinea pigs and NHPs. In guinea pig studies, animals were given one dose of mAb c13C6, h13F6, or c6D8 individually (totaling 5 mg per animal) at 1 day post-infection (dpi) with 1000×LD50 of guinea pig-adapted EBOV, Mayinga variant (EBOV-M-GPA). Survival and weight loss were monitored over 28 days. Treatment with c13C6 or h13F6 yielded 17% survival (1 of 6 animals) with a mean time to death of 8.4±1.7 and 10.2±1.8 days, respectively. The average weight loss for c13C6 or h13F6-treated animals was 9% and 21% (Table 7). In nonhuman primates, animals were given three doses of mAb c13C6, h13F6, or c6D8, beginning at 24 hours after challenge with the Kikwit variant of EBOV (EBOV-K)(34), and survival was monitored over 28 days. Only c13C6 treatment yielded any survivors, with 1 of 3 animals protected from EBOV challenge (Table 7), confirming in two separate animal models that c13C6 is the component that provides the highest level of protection in the MB-003 cocktail.

We then tested mAb c13C6 in combination with two of three mAbs from ZMAb in guinea pigs. The individual antibodies composing ZMAb were originally chosen for protection studies based on their in vivo protection of guinea pigs against EBOV-M-GPA (37), and all three possible combinations were tested: ZMapp1 (c13C6+c2G4+c4G7), ZMapp2 (c13C6+c1H3+c2G4) and ZMapp3 (c13C6+c1H3+c4G7), and compared to the originator cocktails ZMAb and MB-003. Three days after challenge with 1000×LD50 of EBOV-M-GPA, the animals received a single combined dose of 5 mg of antibodies. This dosage is purposely given to elicit a suboptimal level of protection with the cZMAb and MB-003 cocktails, such that potential improvements with the optimized mAb combinations can be identified. Of the tested cocktails, ZMapp1 showed the best protection, with 4 of 6 survivors and less than 5% average weight loss (Table 7). ZMapp2 was next with 3 of 6 survivors and 8% average weight loss, and ZMapp3 protected 1 of 6 animals (Table 7). The level of protection afforded by ZMapp3 was not a statistically significant increase over cZMAb (p=0.224, log-rank test compared to ZMAb, χ²=1.479, df=1), and showed the same survival rate along with a similar average weight loss (Table 7). As a result, only ZMapp1 and ZMapp2 were carried forward to NHP studies.

TABLE 7
Efficacy of individual and combined monoclonal antibody
treatments in guinea pigs and nonhuman primates

| | | | | | | P value, | |
|---|---|---|---|---|---|---|---|
| Treatment | | Mean time to | | | Weight | compared with | |
| groups, time | Dose | death | No. | Survival | loss | | MB- |
| of treatment | (mg) | (days ± s.d.) | survivors/total | (%) | (%) | cZMAb | 003 |
| Guinea pigs | | | | | | | |
| PBS, 3 dpi | N/A | 7.3 ± 0.5 | 0/4 | 0 | 9% | | |
| cZMAb, 3 dpi | 5 | 11.6 ± 1.8 | 1/6 | 17 | 7% | | |
| MB-003, | 5 | 8.2 ± 1.5 | 0/6 | 0 | 40% | | |
| 3 dpi | | | | | | | |
| ZMapp1, | 5 | 9.0 ± 0.0 | 4/6 | 67 | <5% | 0.190 | 0.0147 |
| 3 dpi | | | | | | | |
| ZMapp2, | 5 | 8.3 ± 0.6 | 3/6 | 50 | 8% | 0.634 | 0.0692 |
| 3 dpi | | | | | | | |
| ZMapp3, | 5 | 8.6 ± 1.1 | 1/6 | 17 | 9% | 0.224 | 0.411 |
| 3 dpi | | | | | | | |
| c13C6, 1 dpi | 5 | 8.4 ± 1.7 | 1/6 | 17 | 9% | | |
| h13F6, 1 dpi | 5 | 10.2 ± 1.8 | 1/6 | 17 | 21% | | |
| c6D8, 1 dpi | 5 | 10.5 ± 2.2 | 0/6 | 0 | 38% | | |
| Nonhuman | | | | | | | |
| primates | | | | | | | |
| PBS, 1 dpi | N/A | 8.4 ± 1.9 | 0/1 | 0 | | | |
| MB-003, | 50 | 14.0 ± 2.8 | 1/3 | 33 | | | |
| 1 dpi | | | | | | | |
| c13C6, 1 dpi | 50 | 9.0 ± 1.4 | 1/3 | 33 | | | |
| h13F6, 1 dpi | 50 | 9.0 ± 2.0 | 0/3 | 0 | | | |
| c6D8, 1 dpi | 50 | 9.7 ± 0.6 | 0/3 | 0 | | | |

### Example 3. Deciding Between ZMapp1 or ZMapp2 Using Non-Human Primates (NHPs)

Rhesus macaques were used to determine whether administration of ZMapp1 or ZMapp2 was superior to ZMAb and MB-003 in terms of extending the treatment window. The experiment consisted of six NHPs per group receiving three doses of ZMapp1 or ZMapp2 at 50 mg/kg intravenously (IV) at 3-day intervals, beginning 3 days after a lethal intramuscular (IM) challenge with 4000×TCID₅₀ (or 2512 PFU) of EBOV-K. Control animals were given phosphate-buffered saline (PBS) or mAb 4E10. Mock-treated animals succumbed to disease between 6-7 dpi with symptoms typical of EBOV, characterized by high clinical scores but no fever, in addition to viral titers up to ˜10⁸ and ˜10⁹ TCID₅₀ by the time of death.

All six ZMapp1 treated NHPs survived the challenge with mild signs of disease (p=0.0039, log-rank test, χ²=8.333, df=1), comparing to control animals. A fever was detected in all but one of the NHPs at 3 dpi, the start of the first ZMapp1 dose. Viremia was also detected beginning at 3 dpi by TCID₅₀ in all but one animal from blood sampled just before the administration of the treatment, and similar results were observed by RT-qPCR. The viremia decreased to undetectable levels by 21 dpi. EBOV shedding was not detected from oral, nasal and rectal swabs by RT-qPCR in any of the ZMapp1 treated animals.

TABLE 8
Clinical findings of EBOV-infected NHPs from 1 to 27 dpi

| | | Clinical findings | | | | | |
|---|---|---|---|---|---|---|---|
| Animal | | Body | | White blood | | | |
| ID | Treatment group | temp. | Rash | cells | Platelets | Biochemistry | Outcome |
| A1 | 50 mg kg ¹c13C6 + c2G4 + m4G7, | Fever (6, | | | Thrombocytopenia | ALT↑ (9, | Survived |
| | 3 dpi | 9, 14 dpi) | | | (6, 9 dpi) | 14 dpi), | |
| | | | | | | TBIL↑ | |
| | | | | | | (9 dpi), | |
| | | | | | | PHOS↓ | |
| | | | | | | (6 dpi) | |
| A2 | 50 mg kg ¹c13C6 + c2G4 + m4G7, | Fever | | Leukocytosis | | CRE↓ | Survived |
| | 3 dpi | (3 dpi) | | (3 dpi) | | (14 dpi) | |
| A3 | 50 mg kg ¹c13C6 + c2G4 + m4G7, | Fever | | Leukocytosis | Thrombocytopenia | | Survived |
| | 3 dpi | (3 dpi) | | (3 dpi) | (6 dpi) | | |
| A4 | 50 mg kg ¹c13C6 + c2G4 + m4G7, | | | Leukocytopenia | Thrombocytopenia | | Survived |
| | 3 dpi | | | (9 dpi) | (3, 6, 14, | | |
| | | | | | 21, 27 dpi) | | |
| A5 | 50 mg kg ¹c13C6 + c2G4 + m4G7, | Fever (3, | | Leukocytopenia | Thrombocytopenia | | Survived |
| | 3 dpi | 6, 9 dpi) | | (9 dpi) | (3, 21 dpi) | | |
| A6 | 50 mg kg ¹c13C6 + c2G4 + m4G7, | Fever | | | | | Survived |
| | 3 dpi | (3 dpi) | | | | | |
| B1 | 50 mg kg ¹ ZMapp2, 3 dpi | Fever (3, | | Leukocytopenia | Thrombocytopenia | | Survived |
| | | 14, 21 dpi) | | (6, 14, | (6 dpi) | | |
| | | | | 21, 27 dpi) | | | |
| B2 | 50 mg kg ¹ ZMapp2, 3 dpi | Fever (3, | | | Thrombocytopenia | | Survived |
| | | 6 dpi) | | | (6, 9 dpi) | | |
| B3 | 50 mg kg ¹ ZMapp2, 3 dpi | Fever (3, | Severe | | Thrombocytopenia | ALT↑↑↑ | Died, |
| | | 6 dpi), | rash | | (6, 9 dpi) | (9 dpi), | 9 dpi |
| | | Hypothermia | (9 dpi) | | | TBIL↑↑ | |
| | | (9 dpi) | | | | (9 dpi), | |
| | | | | | | BUN↑↑↑ | |
| | | | | | | (9 dpi), | |
| | | | | | | CRE↑↑↑ | |
| | | | | | | (9 dpi), | |
| | | | | | | GLU↓↓ | |
| | | | | | | (9 dpi) | |
| B4 | 50 mg kg ¹ ZMapp2, 3 dpi | Fever (3, | | Leukocytopenia | Thrombocytopenia | | Survived |
| | | 6 dpi) | | (6 dpi) | (6, 27 dpi) | | |
| B5 | 50 mg kg ¹ ZMapp2, 3 dpi | Fever (3, | | Leukocytosis | Thrombocytopenia | | Survived |
| | | 6, 14, | | (3 dpi) | (3, 6 dpi) | | |
| | | 21 dpi) | | | | | |
| B6 | 50 mg kg ¹ ZMapp2, 3 dpi | Fever | | Leukocytosis | Thrombocytopenia | PHOS↓ | Survived |
| | | (3 dpi) | | (3 dpi), | (6 dpi) | (3 dpi), | |
| | | | | Leukocytopenia | | CRE↓ | |
| | | | | (6, 9, 14, | | (27 dpi) | |
| | | | | 21, 27 dpi) | | | |
| C1 | PBS, 3 dpi | | Moderate | Leukocytosis | Thrombocytopenia | ALB↓ | Died, |
| | | | rash | (3 dpi) | (6, 7 dpi) | (7 dpi), | 7 dpi |
| | | | (6 dpi), | | | ALT↑ | |
| | | | Severe | | | (7 dpi), | |
| | | | rash | | | BUN↑ | |
| | | | (7 dpi) | | | (7 dpi) | |
| C2 | Control mAb, 3 dpi | | Severe | Leukocytopenia | Thrombocytopenia | ALP↑ | Died, |
| | | | rash | (6, 7 dpi) | (6, 7 dpi) | (3 dpi), | 6 dpi |
| | | | (6 dpi) | | | ALT↑↑↑ | |
| | | | | | | (6 dpi), | |
| | | | | | | BUN↑ | |
| | | | | | | (6 dpi), | |
| | | | | | | CRE↑↑↑ | |
| | | | | | | (6 dpi) | |

In Table 8, hypothermia was defined as below 35° C. Fever was defined as >1.0° C. higher than baseline. Mild rash was defined as focal areas of petechiae covering <10% of the skin, moderate rash as areas of petechiae covering 10 to 40% of the skin, and severe rash as areas of petechiae and/or ecchymosis covering >40% of the skin. Leukocytopenia and thrombocytopenia were defined as a >30% decrease in numbers of WBCs and platelets, respectively. Leukocytosis and thrombocytosis were defined as a twofold or greater increase in numbers of WBCs and platelets over baseline, where WBC count >11.000. ↑, two- to threefold increase; ↑↑, four- to fivefold increase; ↑↑↑, greater than fivefold increase; ↓, two- to threefold decrease; ↓↓, four- to fivefold decrease; ↓↓↓, greater than fivefold decrease. ALB, albumin; AMY, amylase; TBIL, total bilirubin; BUN, blood urea nitrogen; PHOS, phosphate; CRE, creatinine; GLU, glucose; GLOB, globulin.

For ZMapp2 treated animals, 5 of 6 NHPs survived with one NHP succumbing to disease at 9 dpi (p=0.0039, log-rank test, χ²=8.333, df=1, comparing to control animals). Surviving animals showed only mild signs of disease (Table 8). The moribund animal showed increased clinical scores, in addition to a drastic drop in body temperature shortly before death. All six ZMapp2 treated animals showed fever in addition to viremia at 3 dpi by TCID₅₀ and RT-qPCR. The administration of ZMapp2 at the reported concentrations was unable to effectively control viremia. Virus shedding was also detected from the oral and rectal swabs by RT-qPCR in the moribund NHP. Since ZMapp1 demonstrated superior protection to ZMapp2 in this survival study, ZMapp1 (now trademarked as ZMAPP by MappBio Pharmaceuticals) was carried forward to test the limits of protection conferred by this mAb cocktail in a subsequent investigation.

### Example 4. Post-Exposure Protection of EBOV-Infected Nonhuman Primates with ZMAPP

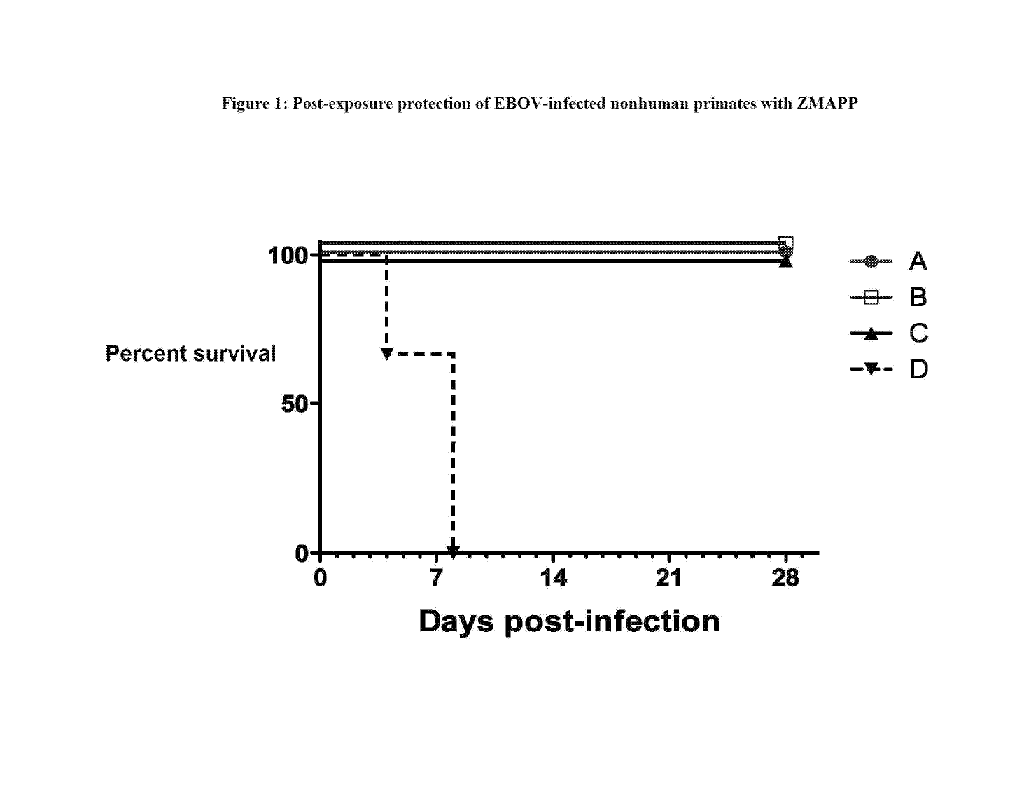

In this experiment, rhesus macaques were assigned into three treatment groups of six and a control group of three animals, with all treatment NHPs receiving three doses of ZMAPP (c13C6+c2G4+c4G7, 50 mg/kg per dose) spaced three days apart. After a lethal IM challenge with 1000×TCID₅₀ (or 628 PFU) of EBOV-K (34), we treated the animals with ZMAPP at 3, 6 and 9 dpi (Group A); 4, 7, and 10 dpi (Group B); or 5, 8 and 11 dpi (Group C). The control animals (Group D) were given mAb 4E10 as an IgG isotype control (n=1) or PBS (n=2) in place of ZMAPP starting at 4 dpi. All animals treated with ZMAPP survived the infection, whereas the three control NHPs (D1, D2 and D3) succumbed to EBOV-K infection at 4, 8 and 8 dpi, respectively (p=3.58E-5, log-rank test, χ2=23.25, df=3, comparing all groups) (FIG. 1). At the time ZMAPP treatment was initiated, fever, leukocytosis, thrombocytopenia and viremia could be detected in the majority of the animals. All animals presented with detectable abnormalities in blood counts and serum biochemistry during the course of the experiment.

Rhesus macaques (n=6 per ZMAPP treatment group, n=3 for controls) were challenged with EBOV-K, and 50 mg/kg of ZMAPP were administered beginning at 3 (Group A), 4 (Group B) or 5 (Group C) days after challenge. Non-specific IgG mAb or PBS was administered as a control (Group D) The Kaplan-Meier survival curves for each group is shown above.

TABLE 9
Clinical findings of EBOV-infected NHPs from 1 to 28 dpi

| | | Clinical findings | | | | | |
|---|---|---|---|---|---|---|---|
| Animal | | Body | | White blood | | | |
| ID | Treatment group | temperature | Rash | cells | Platelets | Biochemistry | Outcome |
| D1 | 50 mg kg ¹ZMapp, | Fever (3, 6, | | Leukocytosis | Thrombocytopenia | ALB↓ (14, | Survived |
| | 3 dpi | 14, 21 dpi) | | (3, 6, 21 dpi) | (3, 6, 9, 14, | 21 dpi), | |
| | | | | | 21 dpi) | ALP↓ (9, 14, | |
| | | | | | | 21, 28 dpi), | |
| | | | | | | AMY↓ | |
| | | | | | | (9 dpi), | |
| | | | | | | GLOB↑ (21, | |
| | | | | | | 28 dpi) | |
| D2 | 50 mg kg ¹ZMapp, | | | Leukocytopenia | Thrombocytopenia | PHOS↓ | Survived |
| | 3 dpi | | | (21, 28 dpi) | (28 dpi) | (9 dpi) | |
| D3 | 50 mg kg ¹ZMapp, | Fever | | Leukocytosis | Thrombocytopenia | ALT↓ | Survived |
| | 3 dpi | (3 dpi) | | (3, 14 dpi) | (3, 21, 28 dpi) | (6 dpi) | |
| D4 | 50 mg kg ¹ZMapp, | | | Leukocytopenia | Thrombocytopenia | ALT↓ | Survived |
| | 3 dpi | | | (14 dpi) | (14, 21 dpi) | (9 dpi), | |
| | | | | | | CRE↑ | |
| | | | | | | (14 dpi) | |
| D5 | 50 mg kg ¹ZMapp, | Fever | | Leukocytopenia | Thrombocytopenia | ALB↓ | Survived |
| | 3 dpi | (3 dpi) | | (21, 28 dpi) | (6, 9 dpi) | (9 dpi), | |
| | | | | | | BUN↓ (3, 6, | |
| | | | | | | 14, 21, | |
| | | | | | | 28 dpi) | |
| D6 | 50 mg kg ¹ZMapp, | | | | Thrombocytopenia | | Survived |
| | 3 dpi | | | | (6 dpi) | | |
| E1 | 50 mg kg ¹ZMapp, | | | | Thrombocytopenia | AMY↓↓ (4, | Survived |
| | 4 dpi | | | | (4, 7, 21 dpi) | 21 dpi), | |
| | | | | | | AMY↓ (7, | |
| | | | | | | 10, 14 dpi), | |
| | | | | | | CRE↓ (21, | |
| | | | | | | 28 dpi) | |
| E2 | 50 mg kg ¹ZMapp, | Fever | | Leukocytosis | Thrombocytopenia | ALT ↓↓ | Survived |
| | 4 dpi | (4 dpi) | | (4, 10 dpi) | (4, 7, 10, 21 dpi) | (4 dpi), | |
| | | | | | | GLU↑ | |
| | | | | | | (4 dpi) | |
| E3 | 50 mg kg ¹ZMapp, | Fever | | Leukocytosis | Thrombocytopenia | CRE↓ | Survived |
| | 4 dpi | (4 dpi) | | (4, 10 dpi) | (7, 10, 14 dpi) | (14 dpi) | |
| E4 | 50 mg kg ¹ZMapp, | | Severe | Leukocytosis | Thrombocytopenia | ALP↑ (7, 10, | Survived |
| | 4 dpi | | rash | (10, 14, 21, | (4, 7, 10, 14 dpi) | 14 dpi), ALT | |
| | | | (5, 6, | 28 dpi) | | ↑↑↑ (7 dpi), | |
| | | | 7, | | | ALT ↑↑ | |
| | | | 8 dpi), | | | (10 dpi), | |
| | | | Mild | | | AMY↓ (4, 7, | |
| | | | rash | | | 10 dpi), | |
| | | | (9 dpi) | | | TBIL↑↑↑ | |
| | | | | | | (7 dpi), | |
| | | | | | | TBIL↑ (10, | |
| | | | | | | 14 dpi), | |
| | | | | | | PHOS↓ (7, | |
| | | | | | | 10 dpi), K⁺↓ | |
| | | | | | | (4 dpi) | |
| E5 | 50 mg kg ¹ZMapp, | Fever | | Leukocytosis | Thrombocytopenia | ALT↑ | Survived |
| | 4 dpi | (7 dpi) | | (4 dpi) | (4, 7, 10, 14 dpi) | (7 dpi), | |
| | | | | | | AMY↓ (4, | |
| | | | | | | 7 dpi), | |
| | | | | | | PHOS↓ | |
| | | | | | | (10 dpi) | |
| E6 | 50 mg kg ¹ZMapp, | Fever | Mild | Leukocytosis | Thrombocytopenia | ALP↑ (7, | Survived |
| | 4 dpi | (4 dpi) | rash | (4, 10, 14 dpi) | (4, 7, 10, 14 dpi) | 10 dpi), ALT | |
| | | | (7, 8, | | | ↑↑↑ (7, 10, | |
| | | | 9 dpi) | | | 14 dpi), | |
| | | | | | | AMY↓ (7, | |
| | | | | | | 10 dpi), | |
| | | | | | | TBIL↑↑ | |
| | | | | | | (7 dpi), | |
| | | | | | | TBIL↑↑↑ | |
| | | | | | | (10 dpi), | |
| | | | | | | TBIL↑ | |
| | | | | | | (14 dpi), | |
| | | | | | | PHOS↓ | |
| | | | | | | (7 dpi), | |
| | | | | | | GLOB↑ | |
| | | | | | | (21 dpi) | |
| F1 | 50 mg kg ¹ZMapp, | | | Leukocytosis | Thrombocytopenia | AMY↓ | Survived |
| | 5 dpi | | | (11 dpi) | (3, 5, 8, 11 dpi) | (5 dpi), | |
| | | | | | | PHOS↓ | |
| | | | | | | (11 dpi), | |
| | | | | | | CRE↓ | |
| | | | | | | (28 dpi) | |
| F2 | 50 mg kg ¹ZMapp, | Fever (3, | Mild | Leukocytosis | Thrombocytopenia | PHOS↓ | Survived |
| | 5 dpi | 5 dpi) | rash | (3, 5, 11 dpi) | (3, 5, 8, 11, 14, | (11 dpi), | |
| | | | (8 dpi) | | 21 dpi) | CRE↓↓ | |
| | | | | | | (11 dpi) | |
| F3 | 50 mg kg ¹ZMapp, | | | Leukocytopenia | Thrombocytopenia | ALT↑ | Survived |
| | 5 dpi | | | (8 dpi), | (5, 8, 11, 21 dpi) | (8 dpi), | |
| | | | | Leukocytosis | | CRE↓↓ | |
| | | | | (3 dpi) | | (14 dpi) | |
| F4 | 50 mg kg ¹ZMapp, | Fever (3, | | Leukocytopenia | Thrombocytopenia | PHOS↓ | Survived |
| | 5 dpi | 5 dpi) | | (8 dpi) | (5, 8, 11, 28 dpi) | (8 dpi) | |
| F5 | 50 mg kg ¹ZMapp, | Fever | | Leukocytosis | Thrombocytopenia | PHOS↓ (5, | Survived |
| | 5 dpi | (3 dpi) | | (3, 11, 14 dpi) | (5, 8, 11 dpi) | 8 dpi), CRE↓ | |
| | | | | | | (8, 11, 21, | |
| | | | | | | 28 dpi) | |
| F6 | 50 mg kg ¹ZMapp, | Fever | | Leukocytopenia | Thrombocytopenia | PHOS↓ (5, | Survived |
| | 5 dpi | (3 dpi) | | (8, 21, | (8, 11, 21 dpi) | 8, 11 dpi), | |
| | | | | 28 dpi) | | GLU↑ | |
| | | | | | | (5 dpi) | |
| G1 | PBS, 4 dpi | | Severe | Leukocytopenia | Thrombocytopenia | AMY↓ | Died, |
| | | | rash | (4 dpi) | (4 dpi) | (4 dpi) | 4 dpi |
| | | | (4 dpi) | | | | |
| G2 | Control mAb, | | Severe | Leukocytopenia | Thrombocytopenia | ALP↑ | Died, |
| | 4 dpi | | rash | (7, 8 dpi) | (4, 7, 8 dpi) | (8 dpi), | 8 dpi |
| | | | (8 dpi) | | | ALT↑ | |
| | | | | | | (7 dpi), ALT | |
| | | | | | | ↑↑↑ (8 dpi), | |
| | | | | | | CRE↑ | |
| | | | | | | (8 dpi) | |
| G3 | PBS, 4 dpi | Fever (4, | Severe | Leukocytopenia | Thrombocytopenia | ALP↑ | Died, |
| | | 8 dpi) | rash | (7, 8 dpi) | (4, 7, 8 dpi) | (8 dpi), | 8 dpi |
| | | | (8 dpi) | | | ALT↑ (7, | |
| | | | | | | 8 dpi), | |
| | | | | | | AMY↓ | |
| | | | | | | (7 dpi), | |
| | | | | | | AMY ↓↓ | |
| | | | | | | (8 dpi), | |
| | | | | | | TBIL↑ | |
| | | | | | | (8 dpi), | |
| | | | | | | PHOS↓ | |
| | | | | | | (7 dpi) | |

In Table 9 hypothermia was defined as below 35° C. Fever was defined as >1.0° C. higher than baseline. Mild rash was defined as focal areas of petechiae covering <10% of the skin, moderate rash was defined as areas of petechiae covering 10 to 40% of the skin, and severe rash was defined as areas of petechiae and/or ecchymosis covering >40% of the skin. Leukocytopenia and thrombocytopenia were defined as a >30% decrease in the numbers of WBCs and platelets, respectively. Leukocytosis and thrombocytosis were defined as a twofold or greater increase in numbers of WBCs and platelets above baseline, where WBC counts are greater than 11.0. ↑, two- to threefold increase; ↑↑, four- to fivefold increase; ↑↑↑, greater than fivefold increase; ↓, two- to threefold decrease; ↓↓, four- to fivefold decrease; ↓↓↓, greater than fivefold decrease. ALB, albumin; ALP, alkaline phosphatase; ALT, alanine aminotransferase; AMY, amylase; TBIL, total bilirubin; BUN, blood urea nitrogen; PHOS, phosphate; CRE, creatinine; GLU, glucose; K⁺, potassium; GLOB, globulin.

In another set of experiments (Table 10) pairs of mAbs were used to treat non-human primates. The combination of 13C6 and 2G4 resulted in an equivalent survival rate compared to ZMAPP.

TABLE 10
Efficacy of pairs of mAbs for treatment of
non-human primates at 3 DPI.

| | Surviving/ | | Mean time | Weight |
|---|---|---|---|---|
| | Total | Survival | to death | loss |
| Treatment groups | animals | (%) | (days) ± SD | (%) |
| ZMapp-N (1:1:1) (n = 8) | 6/8 | 75% | 9.5 ± 2.1 | <5% |
| ZMapp-CHO (1:1:1) | 4/6 | 67% | 13.5 ± 3.5 | <5% |
| (n = 6) | | | | |
| 13C6-N + (2G4 + 4G7)- | 5/6 | 83% | 17 | <5% |
| CHO (1:1:1) (n = 6) | | | | |
| (13C6 + 2G4)-N (1:1) | 2/8 | 25% | 11.7 ± 2.9 | 13% |
| (n = 8) | | | | |
| (13C6 + 2G4)-N (1:2) | 6/8 | 75% | 9.5 ± 0.7 | <5% |
| (n = 8) | | | | |
| (1H3 + 2G4 + 5D2)-CHO | 2/6 | 33% | 15.5 ± 4.4 | 18% |
| (1:1:1) (n = 6) | | | | |
| PBS (n = 4) | 0/4 | 0% | 7.5 ± 0.6 | 26% |
| ZMAb (1:1:1) (n = 4) | 1/4 | 25% | 8 ± 0 | 10% |
| 13C6 + 4G7-N (1:1) | 2/4 | 50% | 7.5 ± 0.7 | <5% |
| (n = 4) | | | | |
| 13C6 + 4G7-N (1:2) | 1/4 | 25% | 9.3 ± 0.6 | <5% |
| (n = 4) | | | | |
| 1H3 + 2G4 (1:1) (n = 4) | 0/4 | 0% | 11.8 ± 3.9 | 31% |
| 1H3 + 4G7 (1:1) (n = 4) | 0/4 | 0% | 8.8 ± 0.5 | 20% |
| 1H3 + 2G4 (1:2) (n = 4) | 1/4 | 25% | 10 ± 1 | 14% |
| 1H3 + 4G7 (1:2) (n = 4) | 0/4 | 0% | 8.5 ± 1.0 | 23% |

Based on clinical scores, the Group F animals in Table 9 did not appear to be as sick as animals E4 and E6, both of whom were near the clinical limit for IACUC mandated euthanasia at 5 and 7 dpi, respectively. Animal E4 had a flushed face and severe rash on more than 40% of its body surface between 5 to 8 dpi in addition to nasal haemorrhage at 7 dpi, whereas animal E6 had a flushed face and petechiae on its arms and legs between 7 to 9 dpi, in addition to jaundice between 10 to 14 dpi. This indicates that host genetic factors may play a role in the differential susceptibility of individual NHPs to EBOV-K infections. Fever, leukocytosis, thrombocytopenia, and a severe rash symptomatic of EBOV disease progression was detected in both E4 and E6 (Table 9). Increases in the level of liver enzymes ALT (10- to 30-fold increase), ALP (2- to 3-fold), and total bilirubin (TBIL, 3- to 11-fold) indicate significant liver damage, a hallmark of filovirus infections. However, ZMAPP was successful in reversing observed disease symptoms and physiological abnormalities after 12 dpi, 2 days after the last ZMAPP administration (Table 9). Furthermore, ZMAPP treatment was able to lower the high virus loads observed in animals F2 and F5 (up to 10⁶ TCID₅₀/ml) to undetectable levels by 14 dpi.

### Example 5. ZMAPP Cross-Reacts with Guinea EBOV

While the results were very promising with EBOV-K infected NHPs, it was unknown whether therapy with ZMAPP would be similarly effective against the Guinean variant of EBOV (EBOV-G), the virus responsible for the West African outbreak. Direct comparison of published amino acid sequences between EBOV-G and EBOV-K showed that the epitopes targeted by ZMAPP (38) (39) were not mutated between the two virus variants, suggesting that the antibodies should retain their specificity for the viral glycoprotein. To confirm this, in vitro assays were carried out to compare the binding affinity of c13C6, c2G4 and c4G7 to sucrose-purified EBOV-G and EBOV-K. As measured by ELISA, the ZMAPP components showed slightly better binding kinetics for EBOV-G than for EBOV-K. Additionally, the neutralizing activity of individual mAbs was evaluated in the absence of complement for c2G4 and c4G7, and in the presence of complement for c13C6, as they have previously been shown to neutralize EBOV only under this condition (28). The results supported the ELISA binding data, with comparable neutralizing activities between the two viruses.

### Example 6. Compassionate Use of ZMAPP on Patients Infected with Ebola

### Study Participants

All patients had a confirmed positive PCR test for Ebola virus prior to administration of the mAbs. Local care givers were responsible for patient selection. Other than symptoms consistent with EVD and virologic diagnosis, the criteria included adult age, severity of symptoms, stage of disease, status as health care workers in an environment critically lacking such personnel, absence of other therapeutic options, patient acceptance of the risk, and drug availability. The proximity of product supply also played a role in patient selection. ZMAPP that was being stored in Africa was used to initiate treatment of the first two patients, and additional doses that had been pre-positioned in the EU under the regulatory authority of SwissMedic were used to treat the third and seventh patients.

Patients received supportive care and additional clinical testing according to the standards and practices of their treating institutions. As such, these measures were not consistent across all patients. Telephonic consultations with prior investigators were incorporated into the preparation for each new patient exposure.

### Study Procedures

At time of use, ZMAPP vials were thawed and diluted in normal saline or Ringers Lactate to a concentration of 4 mg/mL. The prepared solution was either pre-filtered under aseptic conditions through a 0.2 μm, low-protein binding filter, or administered with an in-line 0.2 μm filter. The recommended treatment plan was three doses of 50 mg/kg at three day intervals (i.e., Day 1, Day 4 and Day 7) via intravenous (IV) infusion. For the first infusion, the recommended starting infusion rate was 50 mg/hour (12.5 mL), escalating by 50 mg/hr every 30 minutes up to a maximum rate of 400 mg/hr. Provided that the first infusion was well tolerated, the second and third infusions had a recommended starting at rate of 200 mg/hr, escalating by 200 mg/hr every 15-30 minutes up to a maximum rate of 800 mg/hr. The total duration of infusion ranged from 5 to 20 hours per dose.

All patients were pre-medicated with an antihistamine (diphenhydramine, promethazine or chlorphenamine) prior to receiving each dose of ZMAPP. Administration of these agents was continued at the physicians' discretion during administration. Antipyretics were administered as needed for patient comfort.

Viral load was assessed by quantitative real time reverse transcriptase polymerase chain reaction (qRT-PCR). These assays amplify and detect both positive and negative strand RNA sequences, and do not distinguish between mRNA and viral genomic RNA. For patients 1 & 2 nucleic acid was extracted from 100 μL of undiluted plasma using the Magmax Pathogen RNA/DNA kit (Life Technologies). A qRT-PCR assay targeting the nucleoprotein gene of Ebola virus was used to amplify viral RNA. For patients 3 and 7, nucleic acid amplification tests for detection of EBOV and for quantification of viral load were performed with the use of commercially available kit (Altona; Hamburg, Germany). Standard dilutions were kindly provided by Altona.

Samples were collected and tested during treatment of patients at Emory University Hospital, Royal Free Hospital and Hospital La Paz. Viral load testing protocols for samples collected at Emory University Hospital were conducted by the US Centers for Disease Control (Atlanta, USA) and have been described previously (7). Viral load testing on samples collected at Hospital Universitario La Paz was performed by ISCIII (Madrid, Spain) as described previously (Kreuels B, Wichmann D, Emmerich P, et al. A case of severe Ebola virus infection complicated by gram-negative septicemia. N Engl J Med. 2014. DOI: 0.1056/NEJMoa1411677). Viral load testing of Royal Free samples was performed by Public Health England at the Rare and Imported Pathogens Laboratory (Porton Down, UK). The qRT-PCR assay performed on samples from patients 1 and 2 did not include a concurrently run positive controls to construct a standard curve for precise quantification of RNA copy number. Consequently, Cycle threshold (Ct) values are presented rather than viral RNA copy number. Ct values reflect the number of PCR cycles required to detect the presence of the target sequence with higher Ct values indicating a lower viral load. Samples were considered to be below the assay's limit of detection at a Ct value of >40.

### Viral Load Data

Viral load data are summarized in Table 11. Note that, as these data were generated by different laboratories using different laboratory protocols, the results should not be compared across patients. However, the results do provide a relative indication of changes in viral load within each patient.

TABLE 11
qRT-PCR Results

| | Dose 1 | | Dose 2 | | Dose 3 | |
|---|---|---|---|---|---|---|
| Patient # | Pre | Post | Pre | Post | Pre | Post |
| 1 | — | — | Ct = 26 | Ct = 31.1 | Ct = 32.8 | Ct = 34.8 |
| 2 | — | — | — | — | Ct = 34.9 | Ct = 36 |
| 3 | 1.5 × 10⁶ copies/mL | 2.3 × 10⁵ copies/mL | — | — | — | — |
| 7 | 1.5 × 10⁶ copies/mL | 3.0 × 10⁴ copies/mL | BLOD | BLOD | — | — |

BLOD—Below Limit of Detection

Prior to administration of dose 2, patient 1 had a Ct value of 26. One day after dose 2, the Ct value was 31.1, an ˜32-fold reduction in serum viral RNA. For Patient 2, the Ct values for samples collected before and one day after administration of dose 3 were 34.9 and 36, respectively. The only earlier data available from these patients were collected from samples that preceded ZMAPP administration, and were generated by a different laboratory and protocol. Therefore, those data are not included herein to avoid presenting a false baseline.

Patient 3 had a viral load of 1.5×106 copies/mL serum immediately prior to administration of dose 1. Viral load was 3.6×106 copies/mL serum in the sample collected immediately after administration of this dose, and declined to 2.3×105 copies/mL serum one day after administration of dose 1. Patient 7 had a viral load of 1.5×106 copies/mL serum prior to administration of dose 1. One day after administration, the viral load for this patient was determined to be 3.0×104 copies/mL. Viral load progressively declined below the assay limit of detection immediately prior to administration of dose 2.

### Patient Outcome

Patient outcomes are summarized in Table 12. Of the seven patients who received ZMAPP, five were alive at the time of discharge and two died while hospitalized. Patients who survived were discharged 15-30 days after symptom onset. Patients 3 and 6 died 12 and 26 days after symptom onset, respectively.

TABLE 12
Patient Outcome

| | Number of days after | | | |
|---|---|---|---|---|
| | symptom onset before | Outcome of | | Criteria for discharge and |
| Patient # | ZMapp administration | hospitalization | doi | sequelae/Cause of death |
| 1 | 9 doi | Alive at discharge | 30 | Asymptomatic and PCR- |
| | | | | negative for two |
| | | | | consecutive days. No |
| | | | | significant sequelae. |
| 2 | 10 doi | Alive at discharge | 29 | Asymptomatic and PCR- |
| | | | | negative for two |
| | | | | consecutive days. Sequelae |
| | | | | restricted to a mild |
| | | | | peripheral sensory |
| | | | | neuropathy without motor |
| | | | | involvement. |
| 3 | 9 doi | Death | 12 | Multiple organ failure with |
| | | | | respiratory distress and |
| | | | | severe shock attributed to |
| | | | | progression of EVD |
| 4 | 9 doi | Alive at discharge | 19 | PCR negative and |
| | | | | asymptomatic for 24 hours. |
| | | | | No significant sequelae. |
| 5 | 12 doi | Alive at discharge | 25 | PCR negative and |
| | | | | asymptomatic for 24 hours. |
| | | | | No significant sequelae. |
| 6 | 16 doi | Death | 26 | Progressive neurological |
| | | | | and cognitive impairments |
| | | | | including disorientation, |
| | | | | depression and rapid onset |
| | | | | of stupor attributed to |
| | | | | progression of EVD |
| 7 | 6 doi | Alive at discharge | 15 | Asymptomatic and blood |
| | | | | PCR-negative for six |
| | | | | consecutive days. No |
| | | | | significant sequelae. |

doi = day of illness/date of symptom onset. This is estimated to be 5 days post infection.

Transparent communication of the results from the use of various therapeutic options is critical to developing strategies for treating patients with EVD. ZMAPP has now been safely administered to seven patients following the dosing scheme proven effective in the macaque model.

Importantly, most of this clinical effort (three full courses and two partial courses to five patients) was conducted in West Africa, demonstrating that the product can be considered for use “in the field”. Whereas sophisticated patient monitoring and laboratory capabilities may not be necessary for safe administration, more refined and/or controlled clinical protocols will require a substantial investment in medical and logistical infrastructure in order to provide proof of benefit. The absence of control data and the sparse qRT-PCR data collected in this case series precludes drawing any conclusions about pharmacologic effect. The reductions in viral load from pre- to post-dose in patients 1 (dose 2), 3 (dose 1) and 7 (dose 1) are suggestive, but could also have been influenced by the patients' own immune responses. Sample collection from patients during treatment in Africa was either extremely sparse or not done at all due to the lack of relevant testing equipment and infection control concerns. Immediate viral load testing would permit testing of alternative treatment schemes, including adaptive designs. Early cessation of treatment after achieving blood PCR negativity (as done in patient 7) could significantly reduce the required dosage in light of the supply limitations for this investigational product.

The data that have been reported regarding the use of this monoclonal antibody combination in non-human primate models have been encouraging (see above). However, while Ebola virus infection in NHPs is known to produce a disease with symptoms similar to those in humans, there are clear differences in the experimental system, including nearly universal mortality in the NHPs. The administration of the viral challenge in the NHP experiments was by intramuscular injection of 4,000× the tissue culture infectious dose 50% (TCID50), which probably results in a more rapid disease progression than occurs during a natural infection in humans. Counterbalancing this, initiation of mAb therapy in the reported patients occurred later in the disease course (6-16 days after onset of frank symptoms) than has been explored in NHP studies, where treatment was initiated up to 5 days post-infection, approximately the date of symptom onset.

When the data from compassionate treatment of human patients is combined with the NHP patient data, it is evident that ZMAPP treatment confers superior survival to infected patients. Preferably, treatment with ZMAPP confers survival rates of at least 70%, more preferably survival rates of at least 75% and even more preferably survival rates of 80% or greater when administered at least five days post infection. Survival rates are also impacted by the time of Administration post infection. For example, administration of ZMAPP as much as 14 days post infection to human patients resulted in survival rates of over 70%. If the ZMAPP therapy were to be administered to such patients at an earlier time point, it is expected that the survival rates would approach those seen in the NHP patient studies.

### Discussion

The West African outbreak of 2014 has highlighted the troubling absence of available vaccine or therapeutic options to save thousands of lives and stop the spread of EBOV. The lack of a clinically acceptable treatment offer limited incentive for people who suspect they might be infected to report themselves for medical help. Several previous studies have showed that antibodies are crucial for host survival from EBOV (40) (41) (42). Prior NHP studies have also demonstrated the ZMAb cocktail could protect 100% or 50% of animals when dosing was initiated 1 or 2 dpi, while the MB-003 cocktail protected 67% of animals with the same dosing regimen. Before the success with mAb-based therapies, other candidate therapeutics had only demonstrated efficacy when given within 60 minutes of EBOV exposure.

Our results with ZMAPP, a cocktail comprising of individual mAbs selected from MB-003 and ZMAb, demonstrate for the first time the successful protection of NHPs from EBOV disease when intervention was initiated as late as 5 dpi. In the preceding ZMapp1/ZMapp2 experiment, 11 of 12 treated animals had detectable fever (with the exception of A4), and live virus could be detected in the blood of 11 of 12 animals (with the exception of A3) by 3 dpi. Therefore, for the majority of these animals, treatment was therapeutic (as opposed to post-exposure prophylaxis), initiated after two detectable triggers of disease. ZMapp2 was able to protect 5 of 6 animals when administered at 3 dpi. For reasons currently unknown, the lone non-survivor (B3) experienced a viremia of 10⁶ TCID₅₀ at 3 dpi, which is 100-fold greater than all other NHPs and approximately 10-fold higher than what ZMAb has been reported to suppress in a previous study (31). This indicates enhanced EBOV replication in this animal, possibly due to host factors. It was important to note that despite the high levels of live circulating virus detected in B3, ZMapp2 administration was still able to prolong the life of this animal to 9 dpi, and suggests that in cases of high viremia such as this, the dosage of mAbs should be increased.

The highlight of these experimental results is undoubtedly ZMAPP, which was able to reverse severe EBOV disease as indicated by the elevated liver enzymes, mucosal hemorrhages and rash in animals E4 and E6. The high viremia (up to 10⁶ TCID₅₀/m1 of blood in some animals at the time of intervention) could also be effectively controlled without the presence of escape mutants, leading to full recovery of all treated NHPs by 28 dpi. In the absence of direct evidence demonstrating ZMAPP efficacy against lethal EBOV-G infection in NHPs, results from ELISA and neutralizing antibody assays show that binding specificity is not abrogated between EBOV-K and EBOV-G, and therefore the levels of protection should not be affected. The compassionate use of ZMAPP in two infected American healthcare workers with positive results pertaining to survival and reversion of EBOV disease (43), supports this assertion. Rhesus macaques have approximately 55-80 ml of blood per kg of body weight (44); at a dose of 50 mg/kg of antibodies, the estimated starting concentration is approximately 625-909 μg/ml of blood (total; ˜200-300 μg/ml for each antibody). Therefore, the low EC₅₀ values for EBOV-G (0.004-0.02 μg/ml) bode well for treating EBOV-G infections with ZMAPP.

Since the host antibody response is known to correlate with and is required for protection from EBOV infections (41) (42), mAb-based treatments are likely to form the centerpiece of any future therapeutic strategies for fighting EBOV outbreaks. However, whether ZMAPP-treated survivors can be susceptible to re-infection is unknown. In a previous study of murine ZMAb-treated, EBOV-challenged NHP survivors, a re-challenge of these animals with the same virus at 10 and 13 weeks after initial challenge yielded 6 of 6 survivors and 4 of 6 survivors, respectively (45). While specific CD4⁺ and CD8⁺ T-cell responses could be detected in all animals, the circulating levels of glycoprotein (GP)-specific IgG were shown to be 10-fold lower in non-survivors compared to survivors, suggesting that antibody levels may be indicative of protective immunity (45). Sustained immunity with experimental EBOV vaccines in NHPs remain unknown, however in a recent study, a decrease in GP-specific IgG levels due to old age or a suboptimal reaction to the VSVAG/EBOVGP vaccine in rodents also appear to be indicative of non-survival (46).

ZMAPP consists of a cocktail of highly purified mAbs; which constitutes a less controversial alternative than whole blood transfusions from convalescent survivors, as was performed during the 1995 EBOV outbreak in Kikwit (47). The safety of mAb therapy is well-documented, with generally low rates of adverse reactions, the capacity to confer rapid and specific immunity in all populations, including the young, the elderly and the immunocompromised, and if necessary, the ability to provide higher-than-natural levels of immunity compared to vaccinations (48). The evidence presented here suggests that ZMAPP currently offers the best option of the experimental therapeutics currently in development for treating EBOV-infected patients. We hope that initial safety testing in humans will be undertaken soon, preferably within the next few months, in order to enable the compassionate use of ZMAPP as soon as possible.

In sum, when comparing antibody cocktails that bind to multiple epitopes on the Ebola virus, the most important component of those cocktails in order to achieve complete reversion from lethal Ebola infections in non-human primates is the 13C6 mAb. For example, a cocktail of mAbs consisting of 1H3, 2G4, 4G7 (ZMab (4)), when administered to non-human primates at 48 hours post Ebola infection (EBOV strain Kikwit 95), resulted in a survival rate of 50%. In contrast, the cocktail containing 13C6 (13C6, 2G4, 4G7, ZMapp) when administered to non-human primates up to 5 days post Ebola infection, resulted in 100% survival during the entire course of the study up to 28 days post infection. From these results it can be concluded that the 13C6 mAb contributed an essential binding function that resulted in a survival rate far in excess of the mAb cocktail without 13C6. When compared at equal lower doses (5 mg) in guinea pigs, ZMab resulted in 17% survival whereas ZMAPP resulted in 67% survival (Table 7). Thus, cocktails containing 13C6 are superior other known cocktails or individual monoclonal antibodies, and ZMAPP in particular is vastly more efficacious than other known cocktails for the treatment of Ebola infection.

### Example 7. Isolation and Testing of mAbs Against Marburg Virus

### Isolation of Antibodies

We tested plasma of a MARV survivor previously infected in Uganda for the 50% neutralization activity against the Uganda strain of MARV and found a serum-neutralizing titer of 1:1,010. To generate human hybridoma cell lines secreting mAbs to MARV, we screened supernatants from EBV-transformed B cell lines derived from the survivor for binding to several recombinant forms of MARV GP or to irradiated cell lysates prepared from MARV-infected cell cultures. We fused transformed cells from B cell lines producing MARV-reactive Abs to the MARV antigens with myeloma cells and generated 51 cloned hybridomas secreting MARV-specific human mAbs. Thirty-nine of these mAbs were specific to the MARV GP, while 12 bound to infected-cell lysate, but not to GP; these latter mAbs were shown in secondary screens to bind to MARV internal proteins (NP, VP35, or VP40; data not shown). Analysis of the Ab heavy- and light-chain variable domain sequences revealed that all MARV specific mAbs were encoded by unique Ab genes.

### Neutralization Activity

To evaluate the inhibitory activity of the mAbs, we first performed in vitro neutralization studies using a chimeric vesicular stomatitis virus with MARV GP from Uganda strain on its surface (vesicular stomatitis virus/Marburg glycoprotein recombinant VSV/GP-Uganda). Eighteen of the 39 MARV GP-specific mAbs exhibited neutralization activity against VSV/GP-Uganda. Of those 18 nAbs, 9 displayed strong (IC50<10 mg/ml), 8 nAbs displayed moderate (IC50: 10-99 mg/ml), and one displayed weak (IC50: 100-1,000 mg/ml) neutralizing activity against VSV/GP-Uganda. We also tested the neutralization potency of all nAbs that bound to MARV GP in a plaque reduction assay using live MARV-Uganda virus. Of 18 Abs that neutralized VSV/GP-Uganda, 11 Abs exhibited neutralizing activity against MARV-Uganda. These data suggest that VSV/GP, often used to study neutralizing potency of Abs because of its BSL-2 containment level, is more susceptible to Ab-mediated neutralization than live MARV. This difference is likely explained by the significantly lower copy number of MARV GP molecules that incorporate into VSV particles compared with the large number of GP molecules on the surface of filovirus filaments. Comparison of MARV-neutralizing and non-neutralizing antibodies at concentration up to 1.6 mg/ml revealed dose-dependent activity of those mAbs that neutralized. The neutralization activity of nAbs was not enhanced by the presence of complement. As expected, we did not detect neutralizing activity for any of the 12 Abs specific to MARV NP, VP35, or VP40 proteins.

### Recognition of Varying Forms of GP

To characterize the binding of isolated Abs to recombinant MARV GPs, we performed binding assays using either a recombinant MARV GP ectodomain containing the mucin-like domain (MARV GP) or a recombinant GP lacking residues 257-425 of the mucin-like domain (MARV GPDmuc). Based on OD405 values at the highest Ab concentration tested (Emax) and 50% effective concentration (EC50), we divided the MARV-GP-specific Abs into four major groups, based on binding phenotype. Binding group 1 mAbs had an Emax to GP<2 (i.e., these mAbs never exhibited a maximal binding level to MARV GP); binding group 2 mAbs had an Emax to GP>2, with EC50 for GP<EC50 for GPDmuc (i.e., these mAbs bound to the mucin-like domain or glycan cap); and binding group 3 had an Emax to GP>2, with EC50 for GP>EC50 for GPDmuc (i.e., these mAbs bound equally well to full-length and mucin-deleted forms of GP), with the group 3A mAbs having an EC50 for GP<0.5 mg/ml and the group 3B mAbs having an EC50 for GP>0.5 mg/ml (suggesting that, as a class, the group 3B mAbs possess a lower steady-state KD of binding to GP than did group 3A mAbs). Abs that lacked neutralization activity against VSV/GPUganda or MARV-Uganda fell principally into binding groups 1, 2, and 3A. Interestingly, all VSV/GP-Uganda nAbs displayed a unique binding pattern and segregated into binding group 3B. It was interesting that while both mAbs from groups 3A and 3B bound equally well to the full-length MARV GP and to the GPDmuc, EC50 values for nAbs from binding group 3B were higher than those for non-neutralizing Abs from group 3A.

### Competition-Binding Studies

To determine whether mAbs from distinct binding groups targeted different antigenic regions on the MARV GP surface, we performed a competition-binding assay using a real-time biosensor. We tested 18 MARV nAbs from binding group 3B, 4 Abs from binding group 3A, and 1 Ab from binding group 2 in a tandem blocking assay in which biotinylated GPDmuc was attached to a streptavidin biosensor. Abs from group 1 and the two non-neutralizing Abs from binding group 3B did not bind to biotinylated GPDmuc in the competition assay and were excluded from the analysis. While non-neutralizing Abs from binding groups 2 and 3A did not prevent binding of the binding group 3B nAbs to GPDmuc, all nAbs blocked binding of each of the other nAbs to the antigen and segregated into a single competition-binding group. These data suggested that all of the nAbs target a single major antigenic region on the MARV GP surface.

### In Vivo Testing

We tested the in vivo protective activity of the mAbs in a murine model using mouse-adapted MARV strain. Inoculation of mice with MARV Ci67 causes clinical disease and, in a proportion of animals, causes lethal disease, although typically less than 100% lethality. We selected four of the mAbs among those with the lowest in vitro neutralization IC50 values: MR72, MR82, MR213, and MR232. The IC50 values in neutralization assays with MARV Uganda or mouse-adapted MARV strain Ci67 were comparable (within 2-fold). Seven-week-old BALB/c mice were injected with 100 mg of antibody by the IP route and challenged with 1,000 plaque-forming unit (PFU) of Ci67. Twenty-four hours later, antibody treatment was repeated. By day 6, all five control (untreated) mice developed progressive loss of weight and symptoms of the disease, including dyspnea, recumbency, and unresponsiveness, and on days 8 and 9, two animals were found dead and one animal was found moribund and euthanized. The remaining two animals demonstrated recovery by day 11. In contrast, all animals treated with any antibody survived and did not display the elevation of the disease score, with the exception of two animals treated with MR72, which showed a transient marginal loss of weight and increase of the disease score on days 6-9, which did not exceed 1. The observed level of protection was remarkable given the relatively modest in vitro neutralizing potency of the antibodies.

### Selecting for the Lead Monoclonal

Three of the fully human mAbs against the GP epitope groups were selected for testing in the guinea pig adapted MARV Angola model. Guinea pigs received an intramuscular injection (IM) of 1000 pfu of guinea pig adapted Angola. Challenge with this stock of virus at this titer and via the IM route has resulted in 100% lethality. Two days after infection, treated animals received a 10 mg dose of mAb. MR82-N was significantly less protective (P<0.05 by log-rank) then mAbs MR78-N and MR191-N which provided 100% protection. Viral load in plasma sampled seven days post-infection was undetectable in MR78 and MR191 treated animals while the control animal (1.3×10⁴ pfu/mL) and MR82-N treated animals (mean=5.3×10³ pfu/mL) all had detectable virus.

To select a lead mAb candidate to advance to NHP testing, MR78-N and MR191-N were next tested against guinea pig-adapted MARV (Angola) and RAVV infected animals with a single 10 mg mAb dose four days post-infection. 60% of animals treated with MR78-N and 100% of animals treated with MR191-N survived, with two MR78-N treated animals (5×10¹ and 2.2×10² pfu/mL) and one MR191-N treated animal (100 pfu/mL) having virus detectable by plaque assay in plasma on day 7 post-infection. For comparison, the control animal had a plasma level of 6.3×10⁵ pfu/mL. All animals demonstrated an elevated temperature and approximately half experienced weight loss by day four post-infection, suggesting the treatment with mAb was in a therapeutic context rather than post-exposure prophylaxis. All RAVV infected animals treated four days post-infection with either mAb survived, and none of these animals had detectable virus in plasma on day seven post-infection. In contrast, the control animals both succumbed to infection by ten days post-infection and had plasma viral loads of 1.3 and 1.4×10⁵ pfu/mL. Historic controls infected with this viral stock experienced 100% lethality with a mean time to death of 8-10 days.

Based on these results MR191-N was selected for advancement to NHP testing. However, a final guinea pig experiment was performed testing treatment of MARV Angola infected guinea pigs five days post-infection and 60% (3 of 5) survival was observed with no delay of death in the two animals that succumbed (day 8) compared to the control (day 9).

As observed during the 2014-2015 in West Africa, containment of outbreaks of the filoviruses (Ebola and Marburg) can be challenging, and is made more difficult by lack of approved vaccine or therapeutic options. Here, we show that a single human monoclonal antibody, MR191-N is able to confer a survival benefit of up to 100% to Marburg (Angola) or Raven virus-infected rhesus macaques when treatment is initiated up to 5 days post-infection. High fever, viremia with blood count and chemistry abnormalities were evident in many animals before monoclonal antibody intervention. Advanced disease, as indicated by elevated liver enzymes, mucosal hemorrhages and generalized petechia could be reversed leading to full recovery. These findings extend the growing body of evidence that monoclonal antibodies can have therapeutic benefit during advanced stages of disease with highly virulent viruses.

### REFERENCES

1. Pettitt J, et al. (2013) Therapeutic intervention of Ebola virus infection in rhesus macaques with the MB-003 monoclonal antibody cocktail. Science translational medicine 5(199):199ra113.
2. Qiu X, et al. (2013) Sustained protection against Ebola virus infection following treatment of infected nonhuman primates with ZMAb. Scientific reports 3:3365.
3. Qiu X, et al. (2014) Reversion of advanced Ebola virus disease in nonhuman primates with ZMapp. Nature 514(7520):47-53.
4. Qiu X, et al. (2012) Successful treatment of ebola virus-infected cynomolgus macaques with monoclonal antibodies. Science translational medicine 4(138):138ra181.
5. Hiatt A, Zeitlin L, & Whaley K J (2013) Multiantibody strategies for HIV. Clinical & developmental immunology 2013:632893.
6. Whaley K J, et al. (2012) Emerging Antibody-based Products. Current topics in microbiology and immunology.

Bendandi M, et al. (2010) Rapid, high-yield production in plants of individualized idiotype vaccines for non-Hodgkin's lymphoma. Annals of oncology: official journal of the European Society for Medical Oncology/ESMO 21(12):2420-2427.
8. Castilho A & Steinkellner H (2012) Glyco-engineering in plants to produce human-like N-glycan structures. Biotechnology journal 7(9):1088-1098.
9. Loos A, et al. (2014) Expression and glycoengineering of functionally active heteromultimeric IgM in plants. Proceedings of the National Academy of Sciences of the United States of America.
10. Hiatt A (2014) Designed IgM from glycoengineering. Proceedings of the National Academy of Sciences of the United States of America.
11. Schneider J D, et al. (2014) Oligomerization status influences subcellular deposition and glycosylation of recombinant butyrylcholinesterase in Nicotiana benthamiana. Plant biotechnology journal.
12. Schneider J D, et al. (2014) Expression of human butyrylcholinesterase with an engineered glycosylation profile resembling the plasma-derived orthologue. Biotechnology journal 9(4):501-510.
13. Hiatt A & Pauly M (2006) Monoclonal antibodies from plants: a new speed record. Proceedings of the National Academy of Sciences of the United States of America 103(40):14645-14646.
14. Castilho A, et al. (2010) In planta protein sialylation through overexpression of the respective mammalian pathway. The Journal of biological chemistry 285(21):15923-15930.
15. Guile G R, Rudd P M, Wing D R, Prime S B, & Dwek R A (1996) A rapid high-resolution high-performance liquid chromatographic method for separating glycan mixtures and analyzing oligosaccharide profiles. Analytical biochemistry 240(2):210-226.
16. Bausch D G, Sprecher A G, Jeffs B, & Boumandouki P (2008) Treatment of Marburg and Ebola hemorrhagic fevers: a strategy for testing new drugs and vaccines under outbreak conditions. Antiviral Res 78(1):150-161.
17. Baize S, et al. (2014) Emergence of Zaire Ebola Virus Disease in Guinea—Preliminary Report. N Engl J Med.
18. WHO.int (2014) WHO—Ebola virus disease (EVD).
19. CDC.gov (2014) Chronology of Ebola Hemorrhagic Fever Outbreaks.
20. Reliefweb.int (2014) W. African Ebola epidemic ‘likely to last months’: UN.
21. Clark D V, Jahrling P B, & Lawler J V (2012) Clinical management of filovirus-infected patients. Viruses 4(9):1668-1686.
22. Guimard Y, et al. (1999) Organization of patient care during the Ebola hemorrhagic fever epidemic in Kikwit, Democratic Republic of the Congo, 1995. J Infect Dis 179 Suppl 1:S268-273.
23. Hensley L E, et al. (2007) Recombinant human activated protein C for the postexposure treatment of Ebola hemorrhagic fever. J Infect Dis 196 Suppl 2:S390-399.
24. Geisbert T W, et al. (2003) Treatment of Ebola virus infection with a recombinant inhibitor of factor VIIa/tissue factor: a study in rhesus monkeys. Lancet 362(9400):1953-1958.
25. Geisbert T W, et al. (2010) Postexposure protection of non-human primates against a lethal Ebola virus challenge with RNA interference: a proof-of-concept study. Lancet 375(9729):1896-1905.
26. Warren T K, et al. (2010) Advanced antisense therapies for postexposure protection against lethal Filovirus infections. Nat Med 16(9):991-994.
27. Feldmann H, et al. (2007) Effective post-exposure treatment of Ebola infection. PLoS Pathog 3(1):e2.
28. Olinger G G, Jr., et al. (2012) Delayed treatment of Ebola virus infection with plant-derived monoclonal antibodies provides protection in rhesus macaques. Proc Natl Acad Sci USA 109(44):18030-18035.
29. Qiu X, et al. (2012) Successful treatment of ebola virus-infected cynomolgus macaques with monoclonal antibodies. Sci Transl Med 4(138):138ra181.
30. Pettitt J, et al. (2013) Therapeutic intervention of Ebola virus infection in rhesus macaques with the MB-003 monoclonal antibody cocktail. Sci Transl Med 5(199):199ra113.
31. Qiu X, et al. (2013) mAbs and Ad-Vectored IFN-alpha therapy rescue ebola-infected nonhuman primates when administered after the detection of viremia and symptoms. Sci Transl Med 5(207):207ra143.
32. Giritch A, et al. (2006) Rapid high-yield expression of full-size IgG antibodies in plants coinfected with noncompeting viral vectors. Proc Natl Acad Sci USA 103(40):14701-14706.
33. Zeitlin L, et al. (2011) Enhanced potency of a fucose-free monoclonal antibody being developed as an Ebola virus immunoprotectant. Proc Natl Acad Sci USA 108(51):20690-20694.
   No countermeasures currently exist for the prevention or treatment of the severe sequelae of Filovirus (such as Ebola virus; EBOV) infection. To overcome this limitation in our biodefense preparedness, we have designed monoclonal antibodies (mAbs) which could be used in humans as immunoprotectants for EBOV, starting with a murine mAb (13F6) that recognizes the heavily glycosylated mucin-like domain of the virion-attached glycoprotein (GP). Point mutations were introduced into the variable region of the murine mAb to remove predicted human T-cell epitopes, and the variable regions joined to human constant regions to generate a mAb (h-13F6) appropriate for development for human use. We have evaluated the efficacy of three variants of h-13F6 carrying different glycosylation patterns in a lethal mouse EBOV challenge model. The pattern of glycosylation of the various mAbs was found to correlate to level of protection, with aglycosylated h-13F6 providing the least potent efficacy (ED(50)=33 mug). A version with typical heterogenous mammalian glycoforms (ED(50)=11 mug) had similar potency to the original murine mAb. However, h-13F6 carrying complex N-glycosylation lacking core fucose exhibited superior potency (ED(50)=3 mug). Binding studies using Fcgamma receptors revealed enhanced binding of nonfucosylated h-13F6 to mouse and human FcgammaRIII Together the results indicate the presence of Fc N-glycans enhances the protective efficacy of h-13F6, and that mAbs manufactured with uniform glycosylation and a higher potency glycoform offer promise as biodefense therapeutics.
34. Jahrling P B, et al. (1999) Evaluation of immune globulin and recombinant interferon-alpha2b for treatment of experimental Ebola virus infections. J Infect Dis 179 Suppl 1:S224-234.
35. Connolly B M, et al. (1999) Pathogenesis of experimental Ebola virus infection in guinea pigs. J Infect Dis 179 Suppl 1:S203-217.
36. Reed L J & Muench H (1938) A simple method of estimating fifty percent endpoints. American journal of hygiene 27:493-497.
37. Qiu X, et al. (2012) Ebola GP-specific monoclonal antibodies protect mice and guinea pigs from lethal Ebola virus infection. PLoS Negl Trop Dis 6(3):e1575.
38. Wilson J A, et al. (2000) Epitopes involved in antibody-mediated protection from Ebola virus. Science 287(5458):1664-1666.
39. Qiu X, et al. (2011) Characterization of Zaire ebolavirus glycoprotein-specific monoclonal antibodies. Clin Immunol 141(2):218-227.
40. Dye J M, et al. (2012) Postexposure antibody prophylaxis protects nonhuman primates from filovirus disease. Proc Natl Acad Sci USA 109(13):5034-5039.
41. Wong G, et al. (2012) Immune parameters correlate with protection against ebola virus infection in rodents and nonhuman primates. Sci Transl Med 4(158):158ra146.
42. Marzi A, et al. (2013) Antibodies are necessary for rVSV/ZEBOV-GP-mediated protection against lethal Ebola virus challenge in nonhuman primates. Proc Natl Acad Sci USA 110(5):1893-1898.
43. Promedmail.org (2014) EBOLA VIRUS DISEASE—WEST AFRICA (117): WHO, NIGERIA, LIBERIA, DRUG, MORE.
44. NC3RS.org (2014) Practical blood sample volumes for laboratory animals, domestic species and non-human primates.
45. Qiu X, et al. (2013) Sustained protection against Ebola virus infection following treatment of infected nonhuman primates with ZMAb. Sci Rep 3:3365.
46. Wong G, et al. (2014) Immunization with Vesicular Stomatitis Virus vaccine expressing the Ebola glycoprotein provides sustained long-term protection in rodents. Vaccine in press.
47. Mupapa K, et al. (1999) Treatment of Ebola hemorrhagic fever with blood transfusions from convalescent patients. International Scientific and Technical Committee. J Infect Dis 179 Suppl 1:S18-23.
48. UPMChealthsecurity.org (2013) Next-Generation Monoclonal Antibodies: Challenges and Opportunities. (UPMC Center for Biosecurity).
49. Pogue, G. P., et al. (2010). “Production of pharmaceutical-grade recombinant aprotinin and a monoclonal antibody product using plant-based transient expression systems.” Plant Biotechnol J 8(5): 638-654.
   Plants have been proposed as an attractive alternative for pharmaceutical protein production to current mammalian or microbial cell-based systems. Eukaryotic protein processing coupled with reduced production costs and low risk for mammalian pathogen contamination and other impurities have led many to predict that agricultural systems may offer the next wave for pharmaceutical product production. However, for this to become a reality, the quality of products produced at a relevant scale must equal or exceed the predetermined release criteria of identity, purity, potency and safety as required by pharmaceutical regulatory agencies. In this article, the ability of transient plant virus expression systems to produce a wide range of products at high purity and activity is reviewed. The production of different recombinant proteins is described along with comparisons with established standards, including high purity, specific activity and promising preclinical outcomes. Adaptation of transient plant virus systems to large-scale manufacturing formats required development of virus particle and Agrobacterium inoculation methods. One transient plant system case study illustrates the properties of greenhouse and field-produced recombinant aprotinin compared with an US Food and Drug Administration-approved pharmaceutical product and found them to be highly comparable in all properties evaluated. A second transient plant system case study demonstrates a fully functional monoclonal antibody conforming to release specifications. In conclusion, the production capacity of large quantities of recombinant protein offered by transient plant expression systems, coupled with robust downstream purification approaches, offers a promising solution to recombinant protein production that compares favourably to cell-based systems in scale, cost and quality.
50. Strasser, R., et al. (2008). “Generation of glyco-engineered Nicotiana benthamiana for the production of monoclonal antibodies with a homogeneous human-like N-glycan structure.” Plant Biotechnol J 6(4): 392-402.
   A common argument against using plants as a production system for therapeutic proteins is their inability to perform authentic human N-glycosylation (i.e. the presence of beta1,2-xylosylation and core alpha1,3-fucosylation). In this study, RNA interference (RNAi) technology was used to obtain a targeted down-regulation of the endogenous beta1,2-xylosyltransferase (XylT) and alpha1,3-fucosyltransferase (FucT) genes in Nicotiana benthamiana, a tobacco-related plant species widely used for recombinant protein expression. Three glyco-engineered lines with significantly reduced xylosylated and/or core alpha1,3-fucosylated glycan structures were generated. The human anti HIV monoclonal antibody 2G12 was transiently expressed in these glycosylation mutants as well as in wild-type plants. Four glycoforms of 2G12 differing in the presence/absence of xylose and core alpha1,3-fucose residues in their N-glycans were produced. Notably, 2G12 produced in XylT/FucT-RNAi plants was found to contain an almost homogeneous N-glycan species without detectable xylose and alpha1,3-fucose residues. Plant-derived glycoforms were indistinguishable from Chinese hamster ovary (CHO)-derived 2G12 with respect to electrophoretic properties, and exhibited functional properties (i.e. antigen binding and HIV neutralization activity) at least equivalent to those of the CHO counterpart. The generated RNAi lines were stable, viable and did not show any obvious phenotype, thus providing a robust tool for the production of therapeutically relevant glycoproteins in plants with a humanized N-glycan structure.
51. World Health Organization. Ebola virus disease [Internet]. 2015; Available from: http://www.who.int/mediacentre/factsheets/fs103/en/.
52. Gallagher J. Ebola: Experimental drug ZMapp is “100% effective” in animal trials [Internet]. BBC News2014; Available from: http://www.bbc.com/news/health-28980153
53. Chartrain M, Chu L. Development and production of commercial therapeutic monoclonal antibodies in Mammalian cell expression systems: an overview of the current upstream technologies. Curr Pharm Biotechnol [Internet] 2008 [cited 2015 Aug. 3]; 9:447-67. Available from: http://www.ncbi.nlm.nih.gov/pubmed/19075685
54. Ó'Fágáin C. Engineering protein stability. In: Walls D, Loughran S T, editors. Protein Chromatography. Springer New York Dordrecht Heidelberg London; 2011. page 103-36.
55. Igawa T, Tsunoda H, Kuramochi T, Sampei Z, Ishii S, Hattori K. Engineering the variable region of therapeutic IgG antibodies. MAbs [Internet] 2011; 3:243-52. Available from: http://www.ncbi.nlm.nih.gov/pubmed/21406966.
56. Giudicelli V, Chaume D, Lefranc M-P. IMGT/GENE-DB: a comprehensive database for human and mouse immunoglobulin and T cell receptor genes. Nucleic Acids Res 2005; 33:D256-61.
57. Honegger A M, Plückthun A. Yet another numbering scheme for immunoglobulin variable domains: an automatic modeling and analysis tool. J Mol Biol 2001; 309:657-70.
58. Janda A, Eryilmaz E, Nakouzi A, Cowburn D, Casadevall A. Variable region identical immunoglobulins differing in isotype express different paratopes. J Biol Chem [Internet] 2012; 287:35409-17. Available from: http://www.ncbi.nlm.nih.gov/pubmed/22930758.
59. Buchanan A, Clementel V, Woods R, Ham N, Bowen M a., Mo W, Popovic B, Bishop S M, Dall'Acqua W, Minter R, et al. Engineering a therapeutic IgG molecule to address cysteinylation, aggregation and enhance thermal stability and expression. MAbs 2013; 5:255-62.
60. Yigzaw Y, Hinckley P, Hewig A, Vedantham G. Ion exchange chromatography of proteins and clearance of aggregates. Curr Pharm Biotechnol [Internet] 2009; 10:421-6. Available from: http://www.ncbi.nlm.nih.gov/pubmed/19519418.
61. Lu Y, Williamson B, Gillespie R. Recent advancement in application of hydrophobic interaction chromatography for aggregate removal in industrial purification process. Curr Pharm Biotechnol [Internet] 2009; 10:427-33. Available from: http://www.ncbi.nlm.nih.gov/pubmed/19519419.
62. Gagnon P. Purification Tools for Monoclonal Antibodies. Validated Biosystems Inc; 1996.
63. He F, Phan D H, Hogan S, Bailey R, Becker G W, Narhi L O, Razinkov V I. Detection of IgG aggregation by a high throughput method based on extrinsic fluorescence. J Pharm Sci [Internet] 2010; 99:2598-608. Available from: http://www.ncbi.nlm.nih.gov/pubmed/20039384.
64. Brader M L, Estey T, Bai S, Alston R W, Lucas K K, Lantz S, Landsman P, Maloney K M. Examination of thermal unfolding and aggregation profiles of a series of developable therapeutic monoclonal antibodies. Mol Pharm [Internet] 2015; 12:1005-17. Available from: http://www.ncbi.nlm.nih.gov/pubmed/25687223.
65. Gillespie R, Nguyen T, Macneil S, Jones L, Crampton S, Vunnum S. Cation exchange surface-mediated denaturation of an aglycosylated immunoglobulin (IgG1). J Chromatogr A [Internet] 2012; 1251:101-10. Available from: http://www.ncbi.nlm.nih.gov/pubmed/22771262.
66. Mueller M, Loh M Q, Tscheliessnig R, Tee D H, Tan E, Bardor M, Jungbauer A. Liquid formulations for stabilizing IgMs during physical stress and long-term storage. Pharm Res [Internet] 2013; 30:735-50. Available from: http://www.ncbi.nlm.nih.gov/pubmed/23143394.
67. Chennamsetty N, Voynov V, Kayser V, Helk B, Trout B L. Design of therapeutic proteins with enhanced stability. Proc Natl Acad Sci USA 2009; 106:11937-42.
68. Barsoum J. Introduction of stable high-copy-number DNA into Chinese hamster ovary cells by electroporation. DNA Cell Biol [Internet] 1990 [cited 2015 Aug. 5]; 9:293-300. Available from: http://www.ncbi.nlm.nih.gov/pubmed/2112395.
69. Rogers R S, Nightlinger N S, Livingston B, Campbell P, Bailey R, Balland A. Development of a quantitative mass spectrometry multi-attribute method for characterization, quality control testing and disposition of biologics. MAbs [Internet] 2015; :00-00. Available from: http://www.tandfonline.com/doi/full/10.1080/19420862.2015.1069454.

We claim:

1. A method for the treatment of Ebola in a primate, the method comprising:
a. identifying a primate in need of treatment, and
b. administering a therapeutically effective combination of at least:
i. a first monoclonal antibody comprising a light chain variable region comprising at least one of: an amino acid sequence deduced from the nucleic acid molecule as set forth in SEQ ID NO: 4, chimeric variants thereof, and humanized variants thereof; and a heavy chain variable region comprising at least one of: an amino acid sequence deduced from the nucleic acid molecule as set forth in SEQ. ID NO: 3, chimeric variants thereof, and humanized variants thereof; and
ii. a second monoclonal antibody that binds the Ebola glycoprotein.

2. The method of claim 1, wherein the second monoclonal antibody comprises a light chain variable region comprising at least one of: an amino acid sequence deduced from the nucleic acid molecule as set forth in SEQ ID NO: 6, chimeric variants thereof, and humanized variants thereof; and a heavy chain variable region comprising at least one of: an amino acid sequence deduced from the nucleic acid molecule as set forth in SEQ. ID NO: 5, chimeric variants thereof, and humanized variants thereof.

3. The method of claim 1, wherein the second monoclonal antibody comprises a light chain variable region comprising at least one of: an amino acid sequence deduced from the nucleic acid molecule as set forth in SEQ ID NO: 8, chimeric variants thereof, and humanized variants thereof; and a heavy chain variable region comprising at least one of: an amino acid sequence deduced from the nucleic acid molecule as set forth in SEQ. ID NO: 7, chimeric variants thereof, and humanized variants thereof.

4. The method of claim 1, wherein said humanized variants of said light chain variable region of said first monoclonal antibody comprise the amino acid residues disclosed in at least one of SEQ. ID NO: 18, SEQ. ID NO: 19, and SEQ. ID NO: 20.

5. The method of claim 1, wherein said humanized variants of said heavy chain variable region of said first monoclonal antibody comprise the amino acid residues disclosed in at least one of SEQ. ID NO: 15, SEQ. ID NO: 16, and SEQ. ID NO: 17.

6. The method of claim 1, wherein said humanized variants of said light chain variable region of said first monoclonal antibody comprise the amino acid residues disclosed in at least one of SEQ. ID NO: 24, and SEQ. ID NO: 25.

7. The method of claim 1, wherein said humanized variants of said heavy chain variable region of said first monoclonal antibody comprise the amino acid residues disclosed in at least one of SEQ. ID NO: 21, SEQ. ID NO: 22, and SEQ. ID NO: 23.

8. The method of claim 1, wherein the primate is a human.

9. The method of claim 1, wherein the therapeutically effective combination further comprises: a pharmaceutically acceptable excipient or carrier.

10. The method of claim 1, wherein at least one of the first, and second monoclonal antibodies comprise a predominantly single glycoform.

11. The method of claim 10, wherein the predominantly single glycoform comprises the GnGn glycan.

12. The method of claim 10, wherein the predominantly single glycoform comprises galactosylated glycans.

13. The method of claim 10, wherein the predominantly single glycoform comprises sialylated glycans.

14. The method of claim 10, wherein the predominantly single glycoform comprises less than 5% fucose or xylose.

15. (canceled)

16. (canceled)

17. (canceled)

18. (canceled)

19. (canceled)

20. (canceled)


SEQUENCE LISTING (40 sequences)

SEQ ID NO: 1 - protein, 122 aa, Mus musculus
QLTLKESGPGILKPSQTLSLTCSLSGFSLSTSGVGVGWFRQPSGKGLEWLALIWWDDDKYYNPSLKSQLSISKDFSRNQVFLKISNVDIADTATYYCARRDPFGYDNAMGYWGQGTSVTVSS

SEQ ID NO: 2 - protein, 107 aa, Mus musculus
DIVMTQSQKFMSTSVGDRVSLTCKASQNVGTAVAWYQQKPGQSPKLLIYSASNRYTGVPDRFTGSGSGTDFTLTISNMQSEDLADYFCQQYSSYPLTFGAGTKLELR

SEQ ID NO: 3 - DNA, 366 bp, Mus musculus
cagcttactttgaaagagtccggtccaggaatccttaagccttctcagactttgtctctcacctgttctttgtcaggattctctctttccacttctggagttggagttggttggttcagacaaccttctggaaagggtcttgagtggcttgctctcatctggtgggacgatgacaagtactacaacccaagcttgaagtctcagttgtctatctccaaggacttctctaggaaccaggtgttcttgaagatctccaatgtggacattgccgataccgctacttactattgcgccagaagggacccattcggttacgacaacgctatgggatactggggtcaaggaacttctgtgactgtttcctca

SEQ ID NO: 4 - DNA, 321 bp, Mus musculus
gatattgtgatgacccagagccaaaagttcatgtccacctctgttggtgatagagtgtcacttacttgcaaggcttcccagaatgtgggaactgctgttgcctggtatcaacagaagccaggtcagtctcctaagttgcttatctactcagctagcaaccgttacactggagttccagaccgtttcactggttctggatccggtacagatttcacccttacaatctccaacatgcagtcagaggacttggcagattacttctgccagcagtactcttcctaccctcttacttttggtgcaggaactaagcttgagctcaga

SEQ ID NO: 5 - DNA, 364 bp, Mus musculus
tggaggaggcttgatgcaacctggaggatccatgaaactctcctgtgttgcctcaggattcactttcagtaactactggatgaactgggtccgccagtctccagagaaggggcttgagtgggttgctgaaattagattgaaatctaataattatgcaacacattatgcggagtctgtgaaagggaggttcaccatttcaagagatgattccaaaaggagtgtctacctgcaaatgaataccttaagagctgaagacactggcatttattactgtacccgggggaatggtaactacagggctatggactactggggtcaaggaacctcagtcaccgtctcctcagccaaaacaacacccccatca

SEQ ID NO: 6 - DNA, 306 bp, Mus musculus
gcctccctatctgtatctgtgggagaaactgtctccatcacatgtcgagcaagtgagaatatttacagtagtttagcatggtatcagcagaaacagggaaaatctcctcagctcctggtctattctgcaacaatcttagcagatggtgtgccatcaaggttcagtggcagtggatcaggcactcagtattccctcaagatcaacagcctgcagtctgaagattttgggacttattactgtcaacatttttggggtactccgtacacgttcggaggggggaccaagctggaaataaaacgggctgat

SEQ ID NO: 7 - DNA, 358 bp, Mus musculus
tggacctgagctggagatgcctggcgcttcagtgaagatatcctgcaaggcttctggttcctcattcactggcttcagtatgaactgggtgaagcagagcaatggaaagagccttgagtggattggaaatattgatacttattatggtggtactacctacaaccagaaattcaagggcaaggccacattgactgtggacaaatcctccagcacagcctacatgcagctcaagagcctgacatctgaggactctgcagtctattactgtgcaagatcggcctactacggtagtacttttgcttactggggccaagggactctggtcactgtctctgcagccaaaacaacagccccatcg

SEQ ID NO: 8 - DNA, 306 bp, Mus musculus
gcctccctatctgcatctgtgggagaaactgtcaccatcacatgtcgagcaagtgagaatatttacagttatttagcatggtatcagcagaaacagggaaaatctcctcagctcctggtctataatgccaaaaccttaatagagggtgtgccatcaaggttcagtggcagtggatcaggcacacagttttctctgaagatcaacagcctgcagcctgaagattttgggagttatttctgtcaacatcattttggtactccattcacattcggctcggggacagagttggaaataaaacgggctgat

SEQ ID NO: 9 - protein, 11 aa, Mus musculus
KASQNVGTAVA

SEQ ID NO: 10 - protein, 7 aa, Mus musculus
SASNRYT

SEQ ID NO: 11 - protein, 9 aa, Mus musculus
QQYSSYPLT

SEQ ID NO: 12 - protein, 12 aa, Mus musculus
GFSLSTSGVGVG

SEQ ID NO: 13 - protein, 14 aa, Mus musculus
LIWWDDDKYYNPSL

SEQ ID NO: 14 - protein, 12 aa, Mus musculus
RDPFGYDNAMGY

SEQ ID NO: 15 - protein, 25 aa, Homo sapiens
QVQLKESGPGLLKPSQTLSLTCTVS

SEQ ID NO: 16 - protein, 14 aa, Homo sapiens
WIRQPAGKGLEWIA

SEQ ID NO: 17 - protein, 34 aa, Homo sapiens
KSRLTITKDTSKNQVVLTMTNMDPVDTATYYCAR

SEQ ID NO: 18 - protein, 23 aa, Homo sapiens
DIQMTQSPSFLSASVGDRVTITC

SEQ ID NO: 19 - protein, 15 aa, Homo sapiens
WYQQKPGQPPKLLIY

SEQ ID NO: 20 - protein, 32 aa, Homo sapiens
GVPDRFSGSGSGTDFTLTISSLQAEDVAVYYC

SEQ ID NO: 21 - protein, 25 aa, Homo sapiens
QVQLLESGPGILKPSQTLSLTCSLS

SEQ ID NO: 22 - protein, 25 aa, Homo sapiens
QVQLLESGGGVVKPGQTLSLTCSLS

SEQ ID NO: 23 - protein, 25 aa, Homo sapiens
DVKLLESGGGLVQPGGSLKLSCAAS

SEQ ID NO: 24 - protein, 23 aa, Homo sapiens
DIVMTQSPLSLSTSVGDRVSLTC

SEQ ID NO: 25 - protein, 23 aa, Homo sapiens
DVLLTQIPLSLSTSVGDRVSLTC

SEQ ID NO: 26 - protein, 11 aa, Homo sapiens
FGAGTKLELKR

SEQ ID NO: 27 - protein, 14 aa, Homo sapiens
TGSSSNIGAGFDVH

SEQ ID NO: 28 - protein, 7 aa, Homo sapiens
DNNNRPS

SEQ ID NO: 29 - protein, 12 aa, Homo sapiens
QSYDTSLSGPVV

SEQ ID NO: 30 - protein, 12 aa, Homo sapiens
GVSISDNSYYWG

SEQ ID NO: 31 - protein, 14 aa, Homo sapiens
TISYSGNTYYNPSL

SEQ ID NO: 32 - protein, 16 aa, Homo sapiens
QRIVSGFVEWLSKFDY

SEQ ID NO: 33 - protein, 22 aa, Homo sapiens
QSVLTQPPSVSGAPGQRVTISC

SEQ ID NO: 34 - protein, 15 aa, Homo sapiens
WYQQLPGTAPKLLIY

SEQ ID NO: 35 - protein, 32 aa, Homo sapiens
GVPDRFSGSKSGTSASLAITGLQAEDEADYYC

SEQ ID NO: 36 - protein, 13 aa, Homo sapiens
FGGGTKLTVLQPK

SEQ ID NO: 37 - protein, 25 aa, Homo sapiens
QLQLQESGPGLVKPSETLSLSCTVS

SEQ ID NO: 38 - protein, 14 aa, Homo sapiens
WIRQPPGKGLEWIG

SEQ ID NO: 39 - protein, 34 aa, Homo sapiens
KSRVSISGDTSKHQLSLKVSSVTAADTAVYYCAR

SEQ ID NO: 40 - protein, 11 aa, Homo sapiens
WGQGTLVTVSS